SEQUENCE LISTING (38 sequences)

SEQ ID NO: 1 - DNA, 11337 bp, artificial sequence
  synthetic construct
cgatcgaggaagaggacaagcggctgcttcttaagtttgtgacatcagtatccaaggcaccattgcaaggattcaaggctttgaacccgtcatttgccattcgtaacgctggtagacaggttgatcggttccctacggcctccacctgtgtcaatcttctcaagctgcctgactatcaggacattgatcaacttcggaagaaacttttgtatgccattcgatcacatgctggtttcgatttgtcttagaggaacgcatatacagtaatcatagagaataaacgatattcatttattaaagtagatagttgaggtagaagttgtaaagagtgataaatagcggccgcttactggagctttctggccttctccttggcagcgtcagccttggcctgcttggcgagcttggcgttctttcggtaaaagttgtagaagagaccgagcatggtccacatgtagaaccagagcagagcggtgatgaagaaggggtatccaggtcggccaaggaccttcatggcgtacatgtcccaggaagactggacagacatcatgcagaactgggtcatctgggatcgagtgatgtagaacttgatgaacgacacctgcttgaagcccagggcagacagaaagtagtagccgtacatgatgacgtggatgaaggagttcagggcagcagagaagtaggcttcaccgttgggagcaacgaaggtgaccagccaccagatggtgaagatggaagagtggtggtacacgtgcagaaaggaaatctgtcggttgttcttcttgaggaccatgatcatggtgtcgacaaactccatgatcttggagaagtagaagagccagatcatcttagccatagggagacccttgaaggtgtgatcggcagcgttctcaaacagtccatagttggcctgataagcctcgtacaggatgccaccgcacatgtaggcggagatggagaccagacagaagttgtgcaggagggagaaggtcttgacctcgaatcgttcaaagttcttcatgatctgcatacccacaaacacggtgaccaggtaggcgagcacgatcaggagcacgtggaaggggttcatcagaggcagctctcgagccaggggagactccacggcaaccaggaagcctcgagtgtgatggacaatggtgggaatgtacttctcggcctgggcaaccagggcagcctccaggggatcgacgtagggagcagctcggacaccgatagcgctggcgaggtccatgaacaggtcctgaggcatcttggagggcaggaagggagcaatggactccatgggcaggacctgtgttagtacattgtcggggagtcatcaattggttcgacaggttgtcgactgttagtatgagctcaattgggctctggtgggtcgatgacacttgtcatctgtttctgttgggtcatgtttccatcaccttctatggtactcacaattcgtccgattcgcccgaatccgttaataccgactttgatggccatgttgatgtgtgtttaattcaagaatgaatatagagaagagaagaagaaaaaagattcaattgagccggcgatgcagacccttatataaatgttgccttggacagacggagcaagcccgcccaaacctacgttcggtataatatgttaagctttttaacacaaaggtttggcttggggtaacctgatgtggtgcaaaagaccgggcgttggcgagccattgcgcgggcgaatggggccgtgactcgtctcaaattcgagggcgtgcctcaattcgtgcccccgtggctttttcccgccgtttccgccccgtttgcaccactgcagccgcttctttggttcggacaccttgctgcgagctaggtgccttgtgctacttaaaaagtggcctcccaacaccaacatgacatgagtgcgtgggccaagacacgttggcggggtcgcagtcggctcaatggcccggaaaaaacgctgctggagctggttcggacgcagtccgccgcggcgtatggatatccgcaaggttccatagcgccattgccctccgtcggcgtctatcccgcaacctctaaatagagcgggaatataacccaagcttcttttttttcctttaacacgcacacccccaactatcatgttgctgctgctgtttgactctactctgtggaggggtgctcccacccaacccaacctacaggtggatccggcgctgtgattggctgataagtctcctatccggactaattctgaccaatgggacatgcgcgcaggacccaaatgccgcaattacgtaaccccaacgaaatgcctacccctctttggagcccagcggccccaaatccccccaagcagcccggttctaccggcttccatctccaagcacaagcagcccggttctaccggcttccatctccaagcacccctttctccacaccccacaaaaagacccgtgcaggacatcctactgcgtgtttaaacaccactaaaaccccacaaaatatatcttaccgaatatacagatctactatagaggaacaattgccccggagaagacggccaggccgcctagatgacaaattcaacaactcacagctgactttctgccattgccactaggggggggcctttttatatggccaagccaagctctccacgtcggttgggctgcacccaacaataaatgggtagggttgcaccaacaaagggatgggatggggggtagaagatacgaggataacggggctcaatggcacaaataagaacgaatactgccattaagactcgtgatccagcgactgacaccattgcatcatctaagggcctcaaaactacctcggaactgctgcgctgatctggacaccacagaggttccgagcactttaggttgcaccaaatgtcccaccaggtgcaggcagaaaacgctggaacagcgtgtacagtttgtcttaacaaaaagtgagggcgctgaggtcgagcagggtggtgtgacttgttatagcctttagagctgcgaaagcgcgtatggatttggctcatcaggccagattgagggtctgtggacacatgtcatgttagtgtacttcaatcgccccctggatatagccccgacaataggccgtggcctcatttttttgccttccgcacatttccattgctcggtacccacaccttgcttctcctgcacttgccaaccttaatactggtttacattgaccaacatcttacaagcggggggcttgtctagggtatatataaacagtggctctcccaatcggttgccagtctcttttttcctttctttccccacagattcgaaatctaaactacacatcacacaatgcctgttactgacgtccttaagcgaaagtccggtgtcatcgtcggcgacgatgtccgagccgtgagtatccacgacaagatcagtgtcgagacgacgcgttttgtgtaatgacacaatccgaaagtcgctagcaacacacactctctacacaaactaacccagctctccatggctgccgctccctctgtgcgaacctttacccgagccgaggttctgaacgctgaggctctgaacgagggcaagaaggacgctgaggctcccttcctgatgatcatcgacaacaaggtgtacgacgtccgagagttcgtccctgaccatcctggaggctccgtgattctcacccacgttggcaaggacggcaccgacgtctttgacacctttcatcccgaggctgcttgggagactctcgccaacttctacgttggagacattgacgagtccgaccgagacatcaagaacgatgactttgccgctgaggtccgaaagctgcgaaccctgttccagtctctcggctactacgactcctctaaggcctactacgccttcaaggtctccttcaacctctgcatctggggactgtccaccgtcattgtggccaagtggggtcagacctccaccctcgccaacgtgctctctgctgccctgctcggcctgttctggcagcagtgcggatggctggctcacgactttctgcaccaccaggtcttccaggaccgattctggggtgatctcttcggagccttcctgggaggtgtctgccagggcttctcctcttcctggtggaaggacaagcacaacactcaccatgccgctcccaacgtgcatggcgaggatcctgacattgacacccaccctctcctgacctggtccgagcacgctctggagatgttctccgacgtccccgatgaggagctgacccgaatgtggtctcgattcatggtcctgaaccagacctggttctacttccccattctctccttcgctcgactgtcttggtgcctccagtccattctctttgtgctgcccaacggtcaggctcacaagccctccggagctcgagtgcccatctccctggtcgagcagctgtccctcgccatgcactggacctggtacctcgctaccatgttcctgttcatcaaggatcctgtcaacatgctcgtgtacttcctggtgtctcaggctgtgtgcggaaacctgctcgccatcgtgttctccctcaaccacaacggtatgcctgtgatctccaaggaggaggctgtcgacatggatttctttaccaagcagatcatcactggtcgagatgtccatcctggactgttcgccaactggttcaccggtggcctgaactaccagatcgagcatcacctgttcccttccatgcctcgacacaacttctccaagatccagcctgccgtcgagaccctgtgcaagaagtacaacgtccgataccacaccactggtatgatcgagggaactgccgaggtcttctcccgactgaacgaggtctccaaggccacctccaagatgggcaaggctcagtaagcggccgcatgagaagataaatatataaatacattgagatattaaatgcgctagattagagagcctcatactgctcggagagaagccaagacgagtactcaaaggggattacaccatccatatccacagacacaagctggggaaaggttctatatacactttccggaataccgtagtttccgatgttatcaatgggggcagccaggatttcaggcacttcggtgtctcggggtgaaatggcgttcttggcctccatcaagtcgtaccatgtcttcatttgcctgtcaaagtaaaacagaagcagatgaagaatgaacttgaagtgaaggaatttaaatgtaacgaaactgaaatttgaccagatattgtgtccgcggtggagctccagcttttgttccctttagtgagggttaatttcgagcttggcgtaatcatggtcatagctgtttcctgtgtgaaattgttatccgctcacaagcttccacacaacgtacgccaccattctgtctgccgccatgatgctcaagttctctcttaacatgaagcccgccggtgacgctgttgaggctgccgtcaaggagtccgtcgaggctggtatcactaccgccgatatcggaggctcttcctccacctccgaggtcggagacttgttgccaacaaggtcaaggagctgctcaagaaggagtaagtcgtttctacgacgcattgatggaaggagcaaactgacgcgcctgcgggttggtctaccggcagggtccgctagtgtataagactctataaaaagggccctgccctgctaatgaaatgatgatttataatttaccggtgtagcaaccttgactagaagaagcagattgggtgtgtttgtagtggaggacagtggtacgttttggaaacagtcttcttgaaagtgtcttgtctacagtatattcactcataacctcaatagccaagggtgtagtcggtttattaaaggaagggagttgtggctgatgtggatagatatctttaagctggcgactgcacccaacgagtgtggtggtagcttgttactgtatattcggtaagatatattttgtggggttttagtggtgtttggtaggttagtgcttggtatatgagttgtaggcatgacaatttggaaaggggtggactttgggaatattgtgggatttcaataccttagtttgtacagggtaattgttacaaatgatacaaagaactgtatttcttttcatttgttttaattggttgtatatcaagtccgttagacgagctcagtgggcgcgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgctatcggcatcgacaaggtttgggtccctagccgataccgcactacctgagtcacaatcttcggaggtttagtcttccacatagcacgggcaaaagtgcgtatatatacaagagcgtttgccagccacagattttcactccacacaccacatcacacatacaaccacacacatccacaatggaacccgaaactaagaagaccaagactgactccaagaagattgttcttctcggcggcgacttctgtggccccgaggtgattgccgaggccgtcaaggtgctcaagtctgttgctgaggcctccggcaccgagtttgtgtttgaggaccgactcattggaggagctgccattgagaaggagggcgagcccatcaccgacgctactctcgacatctgccgaaaggctgactctattatgctcggtgctgtcggaggcgctgccaacaccgtatggaccactcccgacggacgaaccgacgtgcgacccgagcagggtctcctcaagctgcgaaaggacctgaacctgtacgccaacctgcgaccctgccagctgctgtcgcccaagctcgccgatctctcccccatccgaaacgttgagggcaccgacttcatcattgtccgagagctcgtcggaggtatctactttggagagcgaaaggaggatgacggatctggcgtcgcttccgacaccgagacctactccgttaattaactttggccggaattcctttacctgcaggataacttcgtataatgtatgctatacgaagttatgatctctctcttgagcttttccataacaagttcttctgcctccaggaagtccatgggtggtttgatcatggttttggtgtagtggtagtgcagtggtggtattgtgactggggatgtagttgagaataagtcatacacaagtcagctttcttcgagcctcatataagtataagtagttcaacgtattagcactgtacccagcatctccgtatcgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaattacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggactaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtgacaccggtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggttttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgtcgcctttgccgacaaaaatgtgatcatgatgaaagccagcaatgacgttgcagctgatattgttgtcggccaaccgcgccgaaaacgcagctgtcagacccacagcctccaacgaagaatgtatcgtcaaagtgatccaagcacactcatagttggagtcgtactccaaaggcggcaatgacgagtcagacagatactcgtcgacgcgataacttcgtataatgtatgctatacgaagttatcgtacgatagttagtagacaacaat

SEQ ID NO: 2 - DNA, 13489 bp, artificial sequence
  synthetic construct
gatctactatagaggaacaattgccccggagaagacggccaggccgcctagatgacaaattcaacaactcacagctgactttctgccattgccactaggggggggcctttttatatggccaagccaagctctccacgtcggttgggctgcacccaacaataaatgggtagggttgcaccaacaaagggatgggatggggggtagaagatacgaggataacggggctcaatggcacaaataagaacgaatactgccattaagactcgtgatccagcgactgacaccattgcatcatctaagggcctcaaaactacctcggaactgctgcgctgatctggacaccacagaggttccgagcactttaggttgcaccaaatgtcccaccaggtgcaggcagaaaacgctggaacagcgtgtacagtttgtcttaacaaaaagtgagggcgctgaggtcgagcagggtggtgtgacttgttatagcctttagagctgcgaaagcgcgtatggatttggctcatcaggccagattgagggtctgtggacacatgtcatgttagtgtacttcaatcgccccctggatatagccccgacaataggccgtggcctcatttttttgccttccgcacatttccattgctcggtacccacaccttgcttctcctgcacttgccaaccttaatactggtttacattgaccaacatcttacaagcggggggcttgtctagggtatatataaacagtggctctcccaatcggttgccagtctcttttttcctttctttccccacagattcgaaatctaaactacacatcacacaatgcctgttactgacgtccttaagcgaaagtccggtgtcatcgtcggcgacgatgtccgagccgtgagtatccacgacaagatcagtgtcgagacgacgcgttttgtgtaatgacacaatccgaaagtcgctagcaacacacactctctacacaaactaacccagctctccatggctatcttcgctgagagagactccactctcatctactctgatcctctgatgctccttgccatcattgagcagcgtctcgaccgacttctgcctgtcgaatccgagcgagactgcgttggtctcgccatgcgagaaggcgctttggcacccggaaagcgaatcagacctgtccttctcatgctggctgcccacgaccttggctaccgagacgaactctctggacttctcgacttcgcctgtgctgtcgagatggttcacgcagcctccctgatcctggatgacattccctgcatggacgatgccgagcttcgacgtggccgacctaccatccatcgacagttcggtgaacccgtggctatcctcgcagccgttgctctgctttcacgagccttcggagtcattgctctggcagacggcatctcttcccaggccaagactcaggccgtggctgagcttagccactccgtcggtattcagggtctggttcaaggacagtttctcgatctgaccgaaggaggtcaaccacgatccgctgatgccattcagcttaccaaccacttcaagacttctgccctgttttcggctgccatgcagatggctgccatcattgctggtgctcctctggcatcccgagagaagttgcatcgtttcgctcgagacctcggacaagcctttcagctgctcgacgatctgacagacggccagagcgacactggcaaggatgcccatcaggacgtcggaaagtctaccctggtcaacatgttgggttccaaagcagtcgagaagcgactgagagaccacttgcgacgtgccgatcgacatctcgcttctgcctgtgactccggatacgccacccgacactttgtgcaggcttggttcgacaaaaagctcgcaatggtcggttaagcggccgcatgagaagataaatatataaatacattgagatattaaatgcgctagattagagagcctcatactgctcggagagaagccaagacgagtactcaaaggggattacaccatccatatccacagacacaagctggggaaaggttctatatacactttccggaataccgtagtttccgatgttatcaatgggggcagccaggatttcaggcacttcggtgtctcggggtgaaatggcgttcttggcctccatcaagtcgtaccatgtcttcatttgcctgtcaaagtaaaacagaagcagatgaagaatgaacttgaagtgaaggaatttaaatgtaacgaaactgaaatttgaccagatattgtgtccgcggtggagctccagcttttgttccctttagtgagggttaatttcgagcttggcgtaatcatggtcatagctgtttcctgtgtgaaattgttatccgctcacaagcttccacacaacgtacgccaccattctgtctgccgccatgatgctcaagttctctcttaacatgaagcccgccggtgacgctgttgaggctgccgtcaaggagtccgtcgaggctggtatcactaccgccgatatcggaggctcttcctccacctccgaggtcggagacttgttgccaacaaggtcaaggagctgctcaagaaggagtaagtcgtttctacgacgcattgatggaaggagcaaactgacgcgcctgcgggttggtctaccggcagggtccgctagtgtataagactctataaaaagggccctgccctgctaatgaaatgatgatttataatttaccggtgtagcaaccttgactagaagaagcagattgggtgtgtttgtagtggaggacagtggtacgttttggaaacagtcttcttgaaagtgtcttgtctacagtatattcactcataacctcaatagccaagggtgtagtcggtttattaaaggaagggagttgtggctgatgtggatagatatctttaagctggcgactgcacccaacgagtgtggtggtagcttgttactgtatattcggtaagatatattttgtggggttttagtggtgtttggtaggttagtgcttggtatatgagttgtaggcatgacaatttggaaaggggtggactttgggaatattgtgggatttcaataccttagtttgtacagggtaattgttacaaatgatacaaagaactgtatttcttttcatttgttttaattggttgtatatcaagtccgttagacgagctcagtgggcgcgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgctatcggcatcgacaaggtttgggtccctagccgataccgcactacctgagtcacaatcttcggaggtttagtcttccacatagcacgggcaaaagtgcgtatatatacaagagcgtttgccagccacagattttcactccacacaccacatcacacatacaaccacacacatccacaatggaacccgaaactaagaagaccaagactgactccaagaagattgttcttctcggcggcgacttctgtggccccgaggtgattgccgaggccgtcaaggtgctcaagtctgttgctgaggcctccggcaccgagtttgtgtttgaggaccgactcattggaggagctgccattgagaaggagggcgagcccatcaccgacgctactctcgacatctgccgaaaggctgactctattatgctcggtgctgtcggaggcgctgccaacaccgtatggaccactcccgacggacgaaccgacgtgcgacccgagcagggtctcctcaagctgcgaaaggacctgaacctgtacgccaacctgcgaccctgccagctgctgtcgcccaagctcgccgatctctcccccatccgaaacgttgagggcaccgacttcatcattgtccgagagctcgtcggaggtatctactttggagagcgaaaggaggatgacggatctggcgtcgcttccgacaccgagacctactccgttaattaactttggccggaattcctttacctgcaggataacttcgtataatgtatgctatacgaagttatgatctctctcttgagcttttccataacaagttcttctgcctccaggaagtccatgggtggtttgatcatggttttggtgtagtggtagtgcagtggtggtattgtgactggggatgtagttgagaataagtcatacacaagtcagctttcttcgagcctcatataagtataagtagttcaacgtattagcactgtacccagcatctccgtatcgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaattacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggactaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtgacaccggtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggttttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgtcgcctttgccgacaaaaatgtgatcatgatgaaagccagcaatgacgttgcagctgatattgttgtcggccaaccgcgccgaaaacgcagctgtcagacccacagcctccaacgaagaatgtatcgtcaaagtgatccaagcacactcatagttggagtcgtactccaaaggcggcaatgacgagtcagacagatactcgtcgacgcgataacttcgtataatgtatgctatacgaagttatcgtacgatagttagtagacaacaatcgatcgaggaagaggacaagcggctgcttcttaagtttgtgacatcagtatccaaggcaccattgcaaggattcaaggctttgaacccgtcatttgccattcgtaacgctggtagacaggttgatcggttccctacggcctccacctgtgtcaatcttctcaagctgcctgactatcaggacattgatcaacttcggaagaaacttttgtatgccattcgatcacatgctggtttcgatttgtcttagaggaacgcatatacagtaatcatagagaataaacgatattcatttattaaagtagatagttgaggtagaagttgtaaagagtgataaatagcggccgcttaacgaggtcgctgccacaactctgcaggtcgtggaggagatgcagcggcacgggatcggatagcttgggcagctcctgcagccagaagcaggagcttctcctgcttcgaggtagactgtcgtctgtcccaggcagtctcgccagcaccgtaaaccttcactccgattcgtctgtagacttccttggcagttgcaatagcccaggcagatcgcaagggaagacctgcgaggccagcgctggcagaggcatagtagggttcagcctcggagacgagtcttcgtgccaagttggcaagagcaggtcgatgggctctgtcagcgaagtgcagtcgatcgagtccagcttcctcgagccaggactcaggcaggtagcaacgtccaactcgtgcatcctcgacaatgtctcgagcaatgttggtaagctgaaaggccagaccgaggtcacaagctcgatccagcacggcttcgtctcgaactcccatgatctgagccatcatgagaccaacgactccagcaacgtggtaacagtatcgcagagtgtcctggaaggtctcgtatctagcacctcgaacgtccatagcaaagccttcgagatgatcgaaggcgtatgctggagagatgtcgtgagcaatggcaacctcctggaaggcagcgaaggcaggttcgtgcatctgagctccagcgtaggcctgtcgagtctttcgttcgaggttagcaagtcgctgttgaggtgtctgtgcagagggaacctcaccaggaaagccgagttgctgatcgtcgatgacatcgtcacagtgtcgacaccaagcgtagagcatcaggacagaacgtcgagtcttggcgtcaaagagcttggaagcggtagcgaacgacttggatccaacctccatagtctcgacagcatggtgcaggagagtagggttgtccatgggcaggacctgtgttagtacattgtcggggagtcatcaattggttcgacaggttgtcgactgttagtatgagctcaattgggctctggtgggtcgatgacacttgtcatctgtttctgttgggtcatgtttccatcaccttctatggtactcacaattcgtccgattcgcccgaatccgttaataccgactttgatggccatgttgatgtgtgtttaattcaagaatgaatatagagaagagaagaagaaaaaagattcaattgagccggcgatgcagacccttatataaatgttgccttggacagacggagcaagcccgcccaaacctacgttcggtataatatgttaagctttttaacacaaaggtttggcttggggtaacctgatgtggtgcaaaagaccgggcgttggcgagccattgcgcgggcgaatggggccgtgactcgtctcaaattcgagggcgtgcctcaattcgtgcccccgtggctttttcccgccgtttccgccccgtttgcaccactgcagccgcttctttggttcggacaccttgctgcgagctaggtgccttgtgctacttaaaaagtggcctcccaacaccaacatgacatgagtgcgtgggccaagacacgttggcggggtcgcagtcggctcaatggcccggaaaaaacgctgctggagctggttcggacgcagtccgccgcggcgtatggatatccgcaaggttccatagcgccattgccctccgtcggcgtctatcccgcaacctctaaatagagcgggaatataacccaagcttcttttttttcctttaacacgcacacccccaactatcatgttgctgctgctgtttgactctactctgtggaggggtgctcccacccaacccaacctacaggtggatccggcgctgtgattggctgataagtctcctatccggactaattctgaccaatgggacatgcgcgcaggacccaaatgccgcaattacgtaaccccaacgaaatgcctacccctctttggagcccagcggccccaaatccccccaagcagcccggttctaccggcttccatctccaagcacaagcagcccggttctaccggcttccatctccaagcacccctttctccacaccccacaaaaagacccgtgcaggacatcctactgcgtgtttaaacatcgtggttaatgctgctgtgtgctgtgtgtgtgtgttgtttggcgctcattgttgcgttatgcagcgtacaccacaatattggaagcttattagcctttctattttttcgtttgcaaggcttaacaacattgctgtggagagggatggggatatggaggccgctggagggagtcggagaggcgttttggagcggcttggcctggcgcccagctcgcgaaacgcacctaggaccctttggcacgccgaaatgtgccacttttcagtctagtaacgccttacctacgtcattccatgcgtgcatgtttgcgccttttttcccttgcccttgatcgccacacagtacagtgcactgtacagtggaggttttgggggggtcttagatgggagctaaaagcggcctagcggtacactagtgggattgtatggagtggcatggagcctaggtggagcctgacaggacgcacgaccggctagcccgtgacagacgatgggtggctcctgttgtccaccgcgtacaaatgtttgggccaaagtcttgtcagccttgcttgcgaacctaattcccaattttgtcacttcgcacccccattgatcgagccctaacccctgcccatcaggcaatccaattaagctcgcattgtctgccttgtttagtttggctcctgcccgtttcggcgtccacttgcacaaacacaaacaagcattatatataaggctcgtctctccctcccaaccacactcacttttttgcccgtcttcccttgctaacacaaaagtcaagaacacaaacaaccaccccaacccccttacacacaagacatatctacagcaatggccatggctcacaccactgtcatcggagctggctttggtggactggctctcgccattcgactgcaggctgcaggcgttcccacccgacttctggagcagcgagacaagcctggtggcagagcctacgtgtaccaggaccaaggcttcacctttgatgctggacccactgtcattaccgatccctccgccatcgaagagctcttcgctcttgccggcaagtccatgcgagactacgttgagctgcttcccgttacccctttctaccgactctgctgggagactggcgaggtctttaactacgataacgatcaggctcgactggaagccgagattcggaagttcaatcctgccgacgtggctggctatcagcgattcctcgactactctcgagccgtcttcgcagaaggttacctcaagttgggaaccgttccctttctgtcctttcgagacatgcttcgagccgctcctcagctcgcacgtcttcaggcttggcgatctgtctactccaaggtggccagcttcattgaggatgacaagctgagacaagccttctcctttcactcgttgctcgttggtggcaacccattcgctacttcctctatctacaccctgattcatgcattggagcgagaatggggtgtctggtttcctcgaggtggcacaggagctctggttcagggtatgctcaagctgttccaggacttgggtggaaccctggagctcaacgccagagtctctcacatcgaggccaaggaggctgccatttccgcagtgcacttggaggatggtcgagtcttcgaaactcgagctgttgcctccaacgccgacgtggttcatacctatggcgatcttctcggaagacatcccgctgcagccgctcaggccaaaaagctgaagggcaagcgaatgtcgaactccttgtttgtcctctacttcggactgaaccaccatcacgaccagcttgctcatcacaccgtctgcttcggtcctcgataccgtgagctcattgacgaaatcttcaaccgagatggacttgccgaagacttctctctctaccttcatgctccctgtgtgactgatccctcgcttgcacctcccggatgtggcagctactatgtcctggctcccgttcctcaccttggtacagccgatctcgactggaacgtcgagggtcctcgactgagagaccgaatctttgcctatctcgaagagcactacatgcctggactgcgatctcaactggttactcatcgaatcttcactcccttcgactttcgagatcagctcaatgcctaccaaggttccgcattctcggtggagcccatcttgagacagtctgcttggtttcgacctcacaaccgagactcgcacattcggaatctctatctggtcggtgccggaacccatcccggtgctggcattcctggagtgatcggttctgccaaggctactgcctccctgatgctcgaggatctgcacgcctaagcggccgcattgatgattggaaacacacacatgggttatatctaggtgagagttagttggacagttatatattaaatcagctatgccaacggtaacttcattcatgtcaacgaggaaccagtgactgcaagtaatatagaatttgaccaccttgccattctcttgcactcctttactatatctcatttatttcttatatacaaatcacttcttcttcccagcatcgagctcggaaacctcatgagcaataacatcgtggatctcgtcaatagagggctttttggactccttgctgttggccaccttgtccttgctgtttaaacaccactaaaaccccacaaaatatatcttaccgaatataca

SEQ ID NO: 3 - DNA, 6540 bp, Artificial Sequence
  Plasmid pZKUGPE1S
ggccgcaagtgtggatggggaagtgagtgcccggttctgtgtgcacaattggcaatccaagatggatggattcaacacagggatatagcgagctacgtggtggtgcgaggatatagcaacggatatttatgtttgacacttgagaatgtacgatacaagcactgtccaagtacaatactaaacatactgtacatactcatactcgtacccgggcaacggtttcacttgagtgcagtggctagtgctcttactcgtacagtgtgcaatactgcgtatcatagtctttgatgtatatcgtattcattcatgttagttgcgtacgaggaaactgtctctgaacagaagaaggaggacgtctctgactacgagaactcccagtacaaggagttcctagtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacagtgctaatacgttgaactacttatacttatatgaggctcgaagaaagctgacttgtgtatgacttaattaatcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagtgctcaagctcgtggcagccaagaaaaccaacctgtgtgcttctctggatgttaccaccaccaaggagctcattgagcttgccgataaggtcggaccttatgtgtgcatgatcaaaacccatatcgacatcattgacgacttcacctacgccggcactgtgctccccctcaaggaacttgctcttaagcacggtttcttcctgttcgaggacagaaagttcgcagatattggcaacactgtcaagcaccagtaccggtgtcaccgaatcgccgagtggtccgatatcaccaacgcccacggtgtacccggaaccggaatcgatgcgtatctgtgggacatgtggtcgttgcgccattatgtaagcagcgtgtactcctctgactgtccatatggtttgctccatctcaccctcatcgttttcattgttcacaggcggccacaaaaaaactgtcttctctccttctctcttcgccttagtctactcggaccagttttagtttagcttggcgccactggataaatgagacctcaggccttgtgatgaggaggtcacttatgaagcatgttaggaggtgcttgtatggatagagaagcacccaaaataataagaataataataaaacagggggcgttgtcatttcatatcgtgttttcaccatcaatacacctccaaacaatgcccttcatgtggccagccccaatattgtcctgtagttcaactctatgcagctcgtatcttattgagcaagtaaaactctgtcagccgatattgcccgacccgcgacaagggtcaacaaggtggtgtaaggccttcgcagaagtcaaaactgtgccaaacaaacatctagagtctctttggtgtttctcgcatatatttwatcggctgtcttacgtatttgcgcctcggtaccggactaatttcggatcatccccaatacgctttttcttcgcagctgtcaacagtgtccatgatctatccacctaaatgggtcatatgaggcgtataatttcgtggtgctgataataattcccatatatttgacacaaaacttccccccctagacatacatctcacaatctcacttcttgtgcttctgtcacacatctcctccagctgacttcaactcacacctctgccccagttggtctacagcggtataaggtttctccgcatagaggtgcaccactcctcccgatacttgtttgtgtgacttgtgggtcacgacatatatatctacacacattgcgccaccctttggttcttccagcacaacaaaaacacgacacgctaaccatggagtccattgctcccttcctgccctccaagatgcctcaggacctgttcatggacctcgccagcgctatcggtgtccgagctgctccctacgtcgatcccctggaggctgccctggttgcccaggccgagaagtacattcccaccattgtccatcacactcgaggcttcctggttgccgtggagtctcccctggctcgagagctgcctctgatgaaccccttccacgtgctcctgatcgtgctcgcctacctggtcaccgtgtttgtgggtatgcagatcatgaagaactttgaacgattcgaggtcaagaccttctccctcctgcacaacttctgtctggtctccatctccgcctacatgtgcggtggcatcctgtacgaggcttatcaggccaactatggactgtttgagaacgctgccgatcacaccttcaagggtctccctatggctaagatgatctggctcttctacttctccaagatcatggagtttgtcgacaccatgatcatggtcctcaagaagaacaaccgacagatttcctttctgcacgtgtaccaccactcttccatcttcaccatctggtggctggtcaccttcgttgctcccaacggtgaagcctacttctctgctgccctgaactccttcatccacgtcatcatgtacggctactactttctgtctgccctgggcttcaagcaggtgtcgttcatcaagttctacatcactcgatcccagatgacccagttctgcatgatgtctgtccagtcttcctgggacatgtacgccatgaaggtccttggccgacctggataccccttcttcatcaccgctctgctctggttctacatgtggaccatgctcggtctcttctacaacttttaccgaaagaacgccaagctcgccaagcaggccaaggctgacgctgccaaggagaaggccagaaagctccagtaagc

SEQ ID NO: 4 - DNA, 15973 bp, artificial sequence
  synthetic construct
aattcctttacctgcaggataacttcgtataatgtatgctatacgaagttatgatctctctcttgagcttttccataacaagttcttctgcctccaggaagtccatgggtggtttgatcatggttttggtgtagtggtagtgcagtggtggtattgtgactggggatgtagttgagaataagtcatacacaagtcagctttcttcgagcctcatataagtataagtagttcaacgtattagcactgtacccagcatctccgtatcgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaattacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggactaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtgacaccggtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggttttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgtcgcctttgccgacaaaaatgtgatcatgatgaaagccagcaatgacgttgcagctgatattgttgtcggccaaccgcgccgaaaacgcagctgtcagacccacagcctccaacgaagaatgtatcgtcaaagtgatccaagcacactcatagttggagtcgtactccaaaggcggcaatgacgagtcagacagatactcgtcgacgcgataacttcgtataatgtatgctatacgaagttatcgtacgatagttagtagacaacaatcgatcgaggaagaggacaagcggctgcttcttaagtttgtgacatcagtatccaaggcaccattgcaaggattcaaggctttgaacccgtcatttgccattcgtaacgctggtagacaggttgatcggttccctacggcctccacctgtgtcaatcttctcaagctgcctgactatcaggacattgatcaacttcggaagaaacttttgtatgccattcgatcacatgctggtttcgatttgtcttagaggaacgcatatacagtaatcatagagaataaacgatattcatttattaaagtagatagttgaggtagaagttgtaaagagtgataaatagcggccgcttaacgaggtcgctgccacaactctgcaggtcgtggaggagatgcagcggcacgggatcggatagcttgggcagctcctgcagccagaagcaggagcttctcctgcttcgaggtagactgtcgtctgtcccaggcagtctcgccagcaccgtaaaccttcactccgattcgtctgtagacttccttggcagttgcaatagcccaggcagatcgcaagggaagacctgcgaggccagcgctggcagaggcatagtagggttcagcctcggagacgagtcttcgtgccaagttggcaagagcaggtcgatgggctctgtcagcgaagtgcagtcgatcgagtccagcttcctcgagccaggactcaggcaggtagcaacgtccaactcgtgcatcctcgacaatgtctcgagcaatgttggtaagctgaaaggccagaccgaggtcacaagctcgatccagcacggcttcgtctcgaactcccatgatctgagccatcatgagaccaacgactccagcaacgtggtaacagtatcgcagagtgtcctggaaggtctcgtatctagcacctcgaacgtccatagcaaagccttcgagatgatcgaaggcgtatgctggagagatgtcgtgagcaatggcaacctcctggaaggcagcgaaggcaggttcgtgcatctgagctccagcgtaggcctgtcgagtctttcgttcgaggttagcaagtcgctgttgaggtgtctgtgcagagggaacctcaccaggaaagccgagttgctgatcgtcgatgacatcgtcacagtgtcgacaccaagcgtagagcatcaggacagaacgtcgagtcttggcgtcaaagagcttggaagcggtagcgaacgacttggatccaacctccatagtctcgacagcatggtgcaggagagtagggttgtccatgggcaggacctgtgttagtacattgtcggggagtcatcaattggttcgacaggttgtcgactgttagtatgagctcaattgggctctggtgggtcgatgacacttgtcatctgtttctgttgggtcatgtttccatcaccttctatggtactcacaattcgtccgattcgcccgaatccgttaataccgactttgatggccatgttgatgtgtgtttaattcaagaatgaatatagagaagagaagaagaaaaaagattcaattgagccggcgatgcagacccttatataaatgttgccttggacagacggagcaagcccgcccaaacctacgttcggtataatatgttaagctttttaacacaaaggtttggcttggggtaacctgatgtggtgcaaaagaccgggcgttggcgagccattgcgcgggcgaatggggccgtgactcgtctcaaattcgagggcgtgcctcaattcgtgcccccgtggctttttcccgccgtttccgccccgtttgcaccactgcagccgcttctttggttcggacaccttgctgcgagctaggtgccttgtgctacttaaaaagtggcctcccaacaccaacatgacatgagtgcgtgggccaagacacgttggcggggtcgcagtcggctcaatggcccggaaaaaacgctgctggagctggttcggacgcagtccgccgcggcgtatggatatccgcaaggttccatagcgccattgccctccgtcggcgtctatcccgcaacctctaaatagagcgggaatataacccaagcttcttttttttcctttaacacgcacacccccaactatcatgttgctgctgctgtttgactctactctgtggaggggtgctcccacccaacccaacctacaggtggatccggcgctgtgattggctgataagtctcctatccggactaattctgaccaatgggacatgcgcgcaggacccaaatgccgcaattacgtaaccccaacgaaatgcctacccctctttggagcccagcggccccaaatccccccaagcagcccggttctaccggcttccatctccaagcacaagcagcccggttctaccggcttccatctccaagcacccctttctccacaccccacaaaaagacccgtgcaggacatcctactgcgtgtttaaacatcgtggttaatgctgctgtgtgctgtgtgtgtgtgttgtttggcgctcattgttgcgttatgcagcgtacaccacaatattggaagcttattagcctttctattttttcgtttgcaaggcttaacaacattgctgtggagagggatggggatatggaggccgctggagggagtcggagaggcgttttggagcggcttggcctggcgcccagctcgcgaaacgcacctaggaccctttggcacgccgaaatgtgccacttttcagtctagtaacgccttacctacgtcattccatgcgtgcatgtttgcgccttttttcccttgcccttgatcgccacacagtacagtgcactgtacagtggaggttttgggggggtcttagatgggagctaaaagcggcctagcggtacactagtgggattgtatggagtggcatggagcctaggtggagcctgacaggacgcacgaccggctagcccgtgacagacgatgggtggctcctgttgtccaccgcgtacaaatgtttgggccaaagtcttgtcagccttgcttgcgaacctaattcccaattttgtcacttcgcacccccattgatcgagccctaacccctgcccatcaggcaatccaattaagctcgcattgtctgccttgtttagtttggctcctgcccgtttcggcgtccacttgcacaaacacaaacaagcattatatataaggctcgtctctccctcccaaccacactcacttttttgcccgtcttcccttgctaacacaaaagtcaagaacacaaacaaccaccccaacccccttacacacaagacatatctacagcaatggccatggctcacaccactgtcatcggagctggctttggtggactggctctcgccattcgactgcaggctgcaggcgttcccacccgacttctggagcagcgagacaagcctggtggcagagcctacgtgtaccaggaccaaggcttcacctttgatgctggacccactgtcattaccgatccctccgccatcgaagagctcttcgctcttgccggcaagtccatgcgagactacgttgagctgcttcccgttacccctttctaccgactctgctgggagactggcgaggtctttaactacgataacgatcaggctcgactggaagccgagattcggaagttcaatcctgccgacgtggctggctatcagcgattcctcgactactctcgagccgtcttcgcagaaggttacctcaagttgggaaccgttccctttctgtcctttcgagacatgcttcgagccgctcctcagctcgcacgtcttcaggcttggcgatctgtctactccaaggtggccagcttcattgaggatgacaagctgagacaagccttctcctttcactcgttgctcgttggtggcaacccattcgctacttcctctatctacaccctgattcatgcattggagcgagaatggggtgtctggtttcctcgaggtggcacaggagctctggttcagggtatgctcaagctgttccaggacttgggtggaaccctggagctcaacgccagagtctctcacatcgaggccaaggaggctgccatttccgcagtgcacttggaggatggtcgagtcttcgaaactcgagctgttgcctccaacgccgacgtggttcatacctatggcgatcttctcggaagacatcccgctgcagccgctcaggccaaaaagctgaagggcaagcgaatgtcgaactccttgtttgtcctctacttcggactgaaccaccatcacgaccagcttgctcatcacaccgtctgcttcggtcctcgataccgtgagctcattgacgaaatcttcaaccgagatggacttgccgaagacttctctctctaccttcatgctccctgtgtgactgatccctcgcttgcacctcccggatgtggcagctactatgtcctggctcccgttcctcaccttggtacagccgatctcgactggaacgtcgagggtcctcgactgagagaccgaatctttgcctatctcgaagagcactacatgcctggactgcgatctcaactggttactcatcgaatcttcactcccttcgactttcgagatcagctcaatgcctaccaaggttccgcattctcggtggagcccatcttgagacagtctgcttggtttcgacctcacaaccgagactcgcacattcggaatctctatctggtcggtgccggaacccatcccggtgctggcattcctggagtgatcggttctgccaaggctactgcctccctgatgctcgaggatctgcacgcctaagcggccgcattgatgattggaaacacacacatgggttatatctaggtgagagttagttggacagttatatattaaatcagctatgccaacggtaacttcattcatgtcaacgaggaaccagtgactgcaagtaatatagaatttgaccaccttgccattctcttgcactcctttactatatctcatttatttcttatatacaaatcacttcttcttcccagcatcgagctcggaaacctcatgagcaataacatcgtggatctcgtcaatagagggctttttggactccttgctgttggccaccttgtccttgctgtttaaacaccactaaaaccccacaaaatatatcttaccgaatatacagatctactatagaggaacaattgccccggagaagacggccaggccgcctagatgacaaattcaacaactcacagctgactttctgccattgccactaggggggggcctttttatatggccaagccaagctctccacgtcggttgggctgcacccaacaataaatgggtagggttgcaccaacaaagggatgggatggggggtagaagatacgaggataacggggctcaatggcacaaataagaacgaatactgccattaagactcgtgatccagcgactgacaccattgcatcatctaagggcctcaaaactacctcggaactgctgcgctgatctggacaccacagaggttccgagcactttaggttgcaccaaatgtcccaccaggtgcaggcagaaaacgctggaacagcgtgtacagtttgtcttaacaaaaagtgagggcgctgaggtcgagcagggtggtgtgacttgttatagcctttagagctgcgaaagcgcgtatggatttggctcatcaggccagattgagggtctgtggacacatgtcatgttagtgtacttcaatcgccccctggatatagccccgacaataggccgtggcctcatttttttgccttccgcacatttccattgctcggtacccacaccttgcttctcctgcacttgccaaccttaatactggtttacattgaccaacatcttacaagcggggggcttgtctagggtatatataaacagtggctctcccaatcggttgccagtctcttttttcctttctttccccacagattcgaaatctaaactacacatcacacaatgcctgttactgacgtccttaagcgaaagtccggtgtcatcgtcggcgacgatgtccgagccgtgagtatccacgacaagatcagtgtcgagacgacgcgttttgtgtaatgacacaatccgaaagtcgctagcaacacacactctctacacaaactaacccagctctccatggctatcttcgctgagagagactccactctcatctactctgatcctctgatgctccttgccatcattgagcagcgtctcgaccgacttctgcctgtcgaatccgagcgagactgcgttggtctcgccatgcgagaaggcgctttggcacccggaaagcgaatcagacctgtccttctcatgctggctgcccacgaccttggctaccgagacgaactctctggacttctcgacttcgcctgtgctgtcgagatggttcacgcagcctccctgatcctggatgacattccctgcatggacgatgccgagcttcgacgtggccgacctaccatccatcgacagttcggtgaacccgtggctatcctcgcagccgttgctctgctttcacgagccttcggagtcattgctctggcagacggcatctcttcccaggccaagactcaggccgtggctgagcttagccactccgtcggtattcagggtctggttcaaggacagtttctcgatctgaccgaaggaggtcaaccacgatccgctgatgccattcagcttaccaaccacttcaagacttctgccctgttttcggctgccatgcagatggctgccatcattgctggtgctcctctggcatcccgagagaagttgcatcgtttcgctcgagacctcggacaagcctttcagctgctcgacgatctgacagacggccagagcgacactggcaaggatgcccatcaggacgtcggaaagtctaccctggtcaacatgttgggttccaaagcagtcgagaagcgactgagagaccacttgcgacgtgccgatcgacatctcgcttctgcctgtgactccggatacgccacccgacactttgtgcaggcttggttcgacaaaaagctcgcaatggtcggttaagcggccgcatgagaagataaatatataaatacattgagatattaaatgcgctagattagagagcctcatactgctcggagagaagccaagacgagtactcaaaggggattacaccatccatatccacagacacaagctggggaaaggttctatatacactttccggaataccgtagtttccgatgttatcaatgggggcagccaggatttcaggcacttcggtgtctcggggtgaaatggcgttcttggcctccatcaagtcgtaccatgtcttcatttgcctgtcaaagtaaaacagaagcagatgaagaatgaacttgaagtgaaggaatttaaatgtaacgaaactgaaatttgaccagatattgtgtccgcggtggagctccagcttttgttccctttagtgagggttaatttcgagcttggcgtaatcatggtcatagctgtttcctgtgtgaaattgttatccgctcacaagcttccacacaacgtacgccaccattctgtctgccgccatgatgctcaagttctctcttaacatgaagcccgccggtgacgctgttgaggctgccgtcaaggagtccgtcgaggctggtatcactaccgccgatatcggaggctcttcctccacctccgaggtcggagacttgttgccaacaaggtcaaggagctgctcaagaaggagtaagtcgtttctacgacgcattgatggaaggagcaaactgacgcgcctgcgggttggtctaccggcagggtccgctagtgtataagactctataaaaagggccctgccctgctaatgaaatgatgatttataatttaccggtgtagcaaccttgactagaagaagcagattgggtgtgtttgtagtggaggacagtggtacgttttggaaacagtcttcttgaaagtgtcttgtctacagtatattcactcataacctcaatagccaagggtgtagtcggtttattaaaggaagggagttgtggctgatgtggatagatatctttaagctggcgactgcacccaacgagtgtggtggtagcttgttactgtatattcggtaagatatattttgtggggttttagtggtgtttggtaggttagtgcttggtatatgagttgtaggcatgacaatttggaaaggggtggactttgggaatattgtgggatttcaataccttagtttgtacagggtaattgttacaaatgatacaaagaactgtatttcttttcatttgttttaattggttgtatatcaagtccgttagacgagctcagtgggcgcgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgctatcggcatcgacaaggtttgggtccctagccgataccgcactacctgagtcacaatcttcggaggtttagtcttccacatagcacgggcaaaagtgcgtatatatacaagagcgtttgccagccacagattttcactccacacaccacatcacacatacaaccacacacatccacaatggaacccgaaactaagaagaccaagactgactccaagaagattgttcttctcggcggcgacttctgtggccccgaggtgattgccgaggccgtcaaggtgctcaagtctgttgctgaggcctccggcaccgagtttgtgtttgaggaccgactcattggaggagctgccattgagaaggagggcgagcccatcaccgacgctactctcgacatctgccgaaaggctgactctattatgctcggtgctgtcggaggcgctgccaacaccgtatggaccactcccgacggacgaaccgacgtgcgacccgagcagggtctcctcaagctgcgaaaggacctgaacctgtacgccaacctgcgaccctgccagctgctgtcgcccaagctcgccgatctctcccccatccgaaacgttgagggcaccgacttcatcattgtccgagagctcgtcggaggtatctactttggagagcgaaaggaggatgacggatctggcgtcgcttccgacaccgagacctactccgttaattaacgatgcgtatctgtgggacatgtggtcgttgcgccattatgtaagcagcgtgtactcctctgactgtttaaaccatatggtttgctccatctcaccctcatcgttttcattgttcacaggcggccacaaaaaaactgtcttctctccttctctcttcgccttagtctactcggaccagttttagtttagcttggcgccactggataaatgagacctcaggccttgtgatgaggaggtcacttatgaagcatgttaggaggtgcttgtatggatagagaagcacccaaaataataagaataataataaaacagggggcgttgtcatttcatatcgtgttttcaccatcaatacacctccaaacaatgcccttcatgtggccagccccaatattgtcctgtagttcaactctatgcagctcgtatcttattgagcaagtaaaactctgtcagccgatattgcccgacccgcgacaagggtcaacaaggtggtgtaaggccttcgcagaagtcaaaactgtgccaaacaaacatctagagtctctttggtgtttctcgcatatatttwatcggctgtcttacgtatttgcgcctcggtaccggactaatttcggatcatccccaatacgctttttcttcgcagctgtcaacagtgtccatgatctatccacctaaatgggtcatatgaggcgtataatttcgtggtgctgataataattcccatatatttgacacaaaacttccccccctagacatacatctcacaatctcacttcttgtgcttctgtcacacatctcctccagctgacttcaactcacacctctgccccagttggtctacagcggtataaggtttctccgcatagaggtgcaccactcctcccgatacttgtttgtgtgacttgtgggtcacgacatatatatctacacacattgcgccaccctttggttcttccagcacaacaaaaacacgacacgctaacccatggcttcccagtacgacctgctccttctcggagctggtctggccaacggactcctggctctccgactgaaagccttgcagcctcaactgcgagtcttggttcttgatgctcacgcacacgctggtggcaaccatacctggtgcttccacgaggaagacctctctgctgcccagcatcagtggattgctcccttggtcgcacatcgttggcctcactacgaggttcgatttcccgctctgactagacagctcaactccggttacttctgtgtcacctcggcacgatttgacgaggttctgcgagccactctcggagatgctctgcgactcaaccagaccgtcgcatcctctggtccagaccacgttcagcttgccagcggcgaagtgctccgagctagagccgtcattgatggacgaggttaccaacccgacgctgcccttcagattggatttcagtccttcgttggtcaggagtggcgactgtctcagcctcatcagctcgaaggtcccattctgatggacgctgccgtggatcagcaaggaggctaccgtttcgtctatacacttcctctctcgcccacccgactgctcattgaggacactcactacatcaacgatgcctccttggctacagcacaggctcgacagaacatctgcgactacgccactcgacaaggatggcagctggagaccctgttgcgagaagagcgaggtgctctgcccatcactcttgcaggcgacttcgatcggttttggcatcaccgtgctccctgtgttggactgagagccggtctcttccatcctaccacaggttactcccttccactggctgccaccctcgctgacgccttggctgccgaggctgacttctctcccgaagcactcgctcctcgtattcaccgatttgcccaggctgcctggcgaaagcaaggctttttcagaatgttgaatcgaatgctgtttcttgctgccgagggagatcgaagatggcgagtcatgcagcgtttctacggtctgcccgagggcttgattgcccgattctatgctggacgactcacacttgccgacagagctcggattctcagcggaaagcctcccgttcctgtgctggctgccctccaggccatccttactcatccttctggtcgaagagcttcacgataagcggccgcattgatgattggaaacacacacatgggttatatctaggtgagagttagttggacagttatatattaaatcagctatgccaacggtaacttcattcatgtcaacgaggaaccagtgactgcaagtaatatagaatttgaccaccttgccattctcttgcactcctttactatatctcatttatttcttatatacaaatcacttcttcttcccagcatcgagctcggaaacctcatgagcaataacatcgtggatctcgtcaatagagggctttttggactccttgctgttggccaccttgtccttgctgtttaaactggctcattctgtttcaacgccttg

SEQ ID NO: 5 - DNA, 912 bp, Enterobacteriaceae sp.
  CDS (2)..(907)
catggctatcttcgctgagagagactccactctcatctactctgatcctctgatgctccttgccatcattgagcagcgtctcgaccgacttctgcctgtcgaatccgagcgagactgcgttggtctcgccatgcgagaaggcgctttggcacccggaaagcgaatcagacctgtccttctcatgctggctgcccacgaccttggctaccgagacgaactctctggacttctcgacttcgcctgtgctgtcgagatggttcacgcagcctccctgatcctggatgacattccctgcatggacgatgccgagcttcgacgtggccgacctaccatccatcgacagttcggtgaacccgtggctatcctcgcagccgttgctctgctttcacgagccttcggagtcattgctctggcagacggcatctcttcccaggccaagactcaggccgtggctgagcttagccactccgtcggtattcagggtctggttcaaggacagtttctcgatctgaccgaaggaggtcaaccacgatccgctgatgccattcagcttaccaaccacttcaagacttctgccctgttttcggctgccatgcagatggctgccatcattgctggtgctcctctggcatcccgagagaagttgcatcgtttcgctcgagacctcggacaagcctttcagctgctcgacgatctgacagacggccagagcgacactggcaaggatgcccatcaggacgtcggaaagtctaccctggtcaacatgttgggttccaaagcagtcgagaagcgactgagagaccacttgcgacgtgccgatcgacatctcgcttctgcctgtgactccggatacgccacccgacactttgtgcaggcttggttcgacaaaaagctcgcaatggtcggttaagc

SEQ ID NO: 6 - protein, 302 aa, Enterobacteriaceae sp.
MAIFAERDSTLIYSDPLMLLAIIEQRLDRLLPVESERDCVGLAMREGALAPGKRIRPVLLMLAAHDLGYRDELSGLLDFACAVEMVHAASLILDDIPCMDDAELRRGRPTIHRQFGEPVAILAAVALLSRAFGVIALADGISSQAKTQAVAELSHSVGIQGLVQGQFLDLTEGGQPRSADAIQLTNHFKTSALFSAAMQMAAIIAGAPLASREKLHRFARDLGQAFQLLDDLTDGQSDTGKDAHQDVGKSTLVNMLGSKAVEKRLRDHLRRADRHLASACDSGYATRHFVQAWFDKKLAMVG

SEQ ID NO: 7 - DNA, 989 bp, artificial sequence
  synthetic construct
gatctactatagaggaacaattgccccggagaagacggccaggccgcctagatgacaaattcaacaactcacagctgactttctgccattgccactaggggggggcctttttatatggccaagccaagctctccacgtcggttgggctgcacccaacaataaatgggtagggttgcaccaacaaagggatgggatggggggtagaagatacgaggataacggggctcaatggcacaaataagaacgaatactgccattaagactcgtgatccagcgactgacaccattgcatcatctaagggcctcaaaactacctcggaactgctgcgctgatctggacaccacagaggttccgagcactttaggttgcaccaaatgtcccaccaggtgcaggcagaaaacgctggaacagcgtgtacagtttgtcttaacaaaaagtgagggcgctgaggtcgagcagggtggtgtgacttgttatagcctttagagctgcgaaagcgcgtatggatttggctcatcaggccagattgagggtctgtggacacatgtcatgttagtgtacttcaatcgccccctggatatagccccgacaataggccgtggcctcatttttttgccttccgcacatttccattgctcggtacccacaccttgcttctcctgcacttgccaaccttaatactggtttacattgaccaacatcttacaagcggggggcttgtctagggtatatataaacagtggctctcccaatcggttgccagtctcttttttcctttctttccccacagattcgaaatctaaactacacatcacacaatgcctgttactgacgtccttaagcgaaagtccggtgtcatcgtcggcgacgatgtccgagccgtgagtatccacgacaagatcagtgtcgagacgacgcgttttgtgtaatgacacaatccgaaagtcgctagcaacacacactctctacacaaactaacccagctctc

SEQ ID NO: 8 - DNA, 322 bp, artificial sequence
  synthetic construct
atgagaagataaatatataaatacattgagatattaaatgcgctagattagagagcctcatactgctcggagagaagccaagacgagtactcaaaggggattacaccatccatatccacagacacaagctggggaaaggttctatatacactttccggaataccgtagtttccgatgttatcaatgggggcagccaggatttcaggcacttcggtgtctcggggtgaaatggcgttcttggcctccatcaagtcgtaccatgtcttcatttgcctgtcaaagtaaaacagaagcagatgaagaatgaacttgaagtgaagga

SEQ ID NO: 9 - DNA, 933 bp, Enterobacteriaceae sp.
gacaaccctactctcctgcaccatgctgtcgagactatggaggttggatccaagtcgttcgctaccgcttccaagctctttgacgccaagactcgacgttctgtcctgatgctctacgcttggtgtcgacactgtgacgatgtcatcgacgatcagcaactcggctttcctggtgaggttccctctgcacagacacctcaacagcgacttgctaacctcgaacgaaagactcgacaggcctacgctggagctcagatgcacgaacctgccttcgctgccttccaggaggttgccattgctcacgacatctctccagcatacgccttcgatcatctcgaaggctttgctatggacgttcgaggtgctagatacgagaccttccaggacactctgcgatactgttaccacgttgctggagtcgttggtctcatgatggctcagatcatgggagttcgagacgaagccgtgctggatcgagcttgtgacctcggtctggcctttcagcttaccaacattgctcgagacattgtcgaggatgcacgagttggacgttgctacctgcctgagtcctggctcgaggaagctggactcgatcgactgcacttcgctgacagagcccatcgacctgctcttgccaacttggcacgaagactcgtctccgaggctgaaccctactatgcctctgccagcgctggcctcgcaggtcttcccttgcgatctgcctgggctattgcaactgccaaggaagtctacagacgaatcggagtgaaggtttacggtgctggcgagactgcctgggacagacgacagtctacctcgaagcaggagaagctcctgcttctggctgcaggagctgcccaagctatccgatcccgtgccgctgcatctcctccacgacctgcagagttgtggcagcgacctcgttaagcggcc

SEQ ID NO: 10 - protein, 309 aa, Enterobacteriaceae sp.
MHNPTLLHHAVETMEVGSKSFATASKLFDAKTRRSVLMLYAWCRHCDDVIDDQQLGFPGEVPSAQTPQQRLANLERKTRQAYAGAQMHEPAFAAFQEVAIAHDISPAYAFDHLEGFAMDVRGARYETFQDTLRYCYHVAGVVGLMMAQIMGVRDEAVLDRACDLGLAFQLTNIARDIVEDARVGRCYLPESWLEEAGLDRLHFADRAHRPALANLARRLVSEAEPYYASASAGLAGLPLRSAWAIATAKEVYRRIGVKVYGAGETAWDRRQSTSKQEKLLLLAAGAAQAIRSRAAASPPRPAELWQRPR

SEQ ID NO: 11 - DNA, 1167 bp, artificial sequence
  synthetic construct
acgcagtaggatgtcctgcacgggtctttttgtggggtgtggagaaaggggtgcttggagatggaagccggtagaaccgggctgcttgtgcttggagatggaagccggtagaaccgggctgcttggggggatttggggccgctgggctccaaagaggggtaggcatttcgttggggttacgtaattgcggcatttgggtcctgcgcgcatgtcccattggtcagaattagtccggataggagacttatcagccaatcacagcgccggatccacctgtaggttgggttgggtgggagcacccctccacagagtagagtcaaacagcagcagcaacatgatagttgggggtgtgcgtgttaaaggaaaaaaaagaagcttgggttatattcccgctctatttagaggttgcgggatagacgccgacggagggcaatggcgctatggaaccttgcggatatccatacgccgcggcggactgcgtccgaaccagctccagcagcgttttttccgggccattgagccgactgcgaccccgccaacgtgtcttggcccacgcactcatgtcatgttggtgttgggaggccactttttaagtagcacaaggcacctagctcgcagcaaggtgtccgaaccaaagaagcggctgcagtggtgcaaacggggcggaaacggcgggaaaaagccacgggggcacgaattgaggcacgccctcgaatttgagacgagtcacggccccattcgcccgcgcaatggctcgccaacgcccggtcttttgcaccacatcaggttaccccaagccaaacctttgtgttaaaaagcttaacatattataccgaacgtaggtttgggcgggcttgctccgtctgtccaaggcaacatttatataagggtctgcatcgccggctcaattgaatcttttttcttcttctcttctctatattcattcttgaattaaacacacatcaacatggccatcaaagtcggtattaacggattcgggcgaatcggacgaattgtgagtaccatagaaggtgatggaaacatgacccaacagaaacagatgacaagtgtcatcgacccaccagagcccaattgagctcatactaacagtcgacaacctgtcgaaccaattgatgactccccgacaatgtactaacacaggtcctg

SEQ ID NO: 12 - DNA, 334 bp, artificial sequence
  synthetic construct
tatttatcactctttacaacttctacctcaactatctactttaataaatgaatatcgtttattctctatgattactgtatatgcgttcctctaagacaaatcgaaaccagcatgtgatcgaatggcatacaaaagtttcttccgaagttgatcaatgtcctgatagtcaggcagcttgagaagattgacacaggtggaggccgtagggaaccgatcaacctgtctaccagcgttacgaatggcaaatgacgggttcaaagccttgaatccttgcaatggtgccttggatactgatgtcacaaacttaagaagcagccgcttgtcctcttcctcg

SEQ ID NO: 13 - DNA, 1485 bp, Enterobacteriaceae sp.
catggctcacaccactgtcatcggagctggctttggtggactggctctcgccattcgactgcaggctgcaggcgttcccacccgacttctggagcagcgagacaagcctggtggcagagcctacgtgtaccaggaccaaggcttcacctttgatgctggacccactgtcattaccgatccctccgccatcgaagagctcttcgctcttgccggcaagtccatgcgagactacgttgagctgcttcccgttacccctttctaccgactctgctgggagactggcgaggtctttaactacgataacgatcaggctcgactggaagccgagattcggaagttcaatcctgccgacgtggctggctatcagcgattcctcgactactctcgagccgtcttcgcagaaggttacctcaagttgggaaccgttccctttctgtcctttcgagacatgcttcgagccgctcctcagctcgcacgtcttcaggcttggcgatctgtctactccaaggtggccagcttcattgaggatgacaagctgagacaagccttctcctttcactcgttgctcgttggtggcaacccattcgctacttcctctatctacaccctgattcatgcattggagcgagaatggggtgtctggtttcctcgaggtggcacaggagctctggttcagggtatgctcaagctgttccaggacttgggtggaaccctggagctcaacgccagagtctctcacatcgaggccaaggaggctgccatttccgcagtgcacttggaggatggtcgagtcttcgaaactcgagctgttgcctccaacgccgacgtggttcatacctatggcgatcttctcggaagacatcccgctgcagccgctcaggccaaaaagctgaagggcaagcgaatgtcgaactccttgtttgtcctctacttcggactgaaccaccatcacgaccagcttgctcatcacaccgtctgcttcggtcctcgataccgtgagctcattgacgaaatcttcaaccgagatggacttgccgaagacttctctctctaccttcatgctccctgtgtgactgatccctcgcttgcacctcccggatgtggcagctactatgtcctggctcccgttcctcaccttggtacagccgatctcgactggaacgtcgagggtcctcgactgagagaccgaatctttgcctatctcgaagagcactacatgcctggactgcgatctcaactggttactcatcgaatcttcactcccttcgactttcgagatcagctcaatgcctaccaaggttccgcattctcggtggagcccatcttgagacagtctgcttggtttcgacctcacaaccgagactcgcacattcggaatctctatctggtcggtgccggaacccatcccggtgctggcattcctggagtgatcggttctgccaaggctactgcctccctgatgctcgaggatctgcacgcctaagc

SEQ ID NO: 14 - protein, 493 aa, Enterobacteriaceae sp.
MKHTTVIGAGFGGLALAIRLQAAGVPTRLLEQRDKPGGRAYVYQDQGFTFDAGPTVITDPSAIEELFALAGKSMRDYVELLPVTPFYRLCWETGEVFNYDNDQARLEAEIRKFNPADVAGYQRFLDYSRAVFAEGYLKLGTVPFLSFRDMLRAAPQLARLQAWRSVYSKVASFIEDDKLRQAFSFHSLLVGGNPFATSSIYTLIHALEREWGVWFPRGGTGALVQGMLKLFQDLGGTLELNARVSHIEAKEAAISAVHLEDGRVFETRAVASNADVVHTYGDLLGRHPAAAAQAKKLKGKRMSNSLFVLYFGLNHHHDQLAHHTVCFGPRYRELIDEIFNRDGLAEDFSLYLHAPCVTDPSLAPPGCGSYYVLAPVPHLGTADLDWNVEGPRLRDRIFAYLEEHYMPGLRSQLVTHRIFTPFDFRDQLNAYQGSAFSVEPILRQSAWFRPHNRDSHIRNLYLVGAGTHPGAGIPGVIGSAKATASLMLEDLHA

SEQ ID NO: 15 - DNA, 842 bp, artificial sequence
  synthetic construct
aaacatcgtggttaatgctgctgtgtgctgtgtgtgtgtgttgtttggcgctcattgttgcgttatgcagcgtacaccacaatattggaagcttattagcctttctattttttcgtttgcaaggcttaacaacattgctgtggagagggatggggatatggaggccgctggagggagtcggagaggcgttttggagcggcttggcctggcgcccagctcgcgaaacgcacctaggaccctttggcacgccgaaatgtgccacttttcagtctagtaacgccttacctacgtcattccatgcgtgcatgtttgcgccttttttcccttgcccttgatcgccacacagtacagtgcactgtacagtggaggttttgggggggtcttagatgggagctaaaagcggcctagcggtacactagtgggattgtatggagtggcatggagcctaggtggagcctgacaggacgcacgaccggctagcccgtgacagacgatgggtggctcctgttgtccaccgcgtacaaatgtttgggccaaagtcttgtcagccttgcttgcgaacctaattcccaattttgtcacttcgcacccccattgatcgagccctaacccctgcccatcaggcaatccaattaagctcgcattgtctgccttgtttagtttggctcctgcccgtttcggcgtccacttgcacaaacacaaacaagcattatatataaggctcgtctctccctcccaaccacactcacttttttgcccgtcttcccttgctaacacaaaagtcaagaacacaaacaaccaccccaacccccttacacacaagacatatctacagcaatggc

SEQ ID NO: 16 - DNA, 313 bp, artificial sequence
  synthetic construct
gcattgatgattggaaacacacacatgggttatatctaggtgagagttagttggacagttatatattaaatcagctatgccaacggtaacttcattcatgtcaacgaggaaccagtgactgcaagtaatatagaatttgaccaccttgccattctcttgcactcctttactatatctcatttatttcttatatacaaatcacttcttcttcccagcatcgagctcggaaacctcatgagcaataacatcgtggatctcgtcaatagagggctttttggactccttgctgttggccaccttgtccttgctgttt

SEQ ID NO: 17 - DNA, 1164 bp, Enterobacteriaceae sp.
atggcttcccagtacgacctgctccttctcggagctggtctggccaacggactcctggctctccgactgaaagccttgcagcctcaactgcgagtcttggttcttgatgctcacgcacacgctggtggcaaccatacctggtgcttccacgaggaagacctctctgctgcccagcatcagtggattgctcccttggtcgcacatcgttggcctcactacgaggttcgatttcccgctctgactagacagctcaactccggttacttctgtgtcacctcggcacgatttgacgaggttctgcgagccactctcggagatgctctgcgactcaaccagaccgtcgcatcctctggtccagaccacgttcagcttgccagcggcgaagtgctccgagctagagccgtcattgatggacgaggttaccaacccgacgctgcccttcagattggatttcagtccttcgttggtcaggagtggcgactgtctcagcctcatcagctcgaaggtcccattctgatggacgctgccgtggatcagcaaggaggctaccgtttcgtctatacacttcctctctcgcccacccgactgctcattgaggacactcactacatcaacgatgcctccttggctacagcacaggctcgacagaacatctgcgactacgccactcgacaaggatggcagctggagaccctgttgcgagaagagcgaggtgctctgcccatcactcttgcaggcgacttcgatcggttttggcatcaccgtgctccctgtgttggactgagagccggtctcttccatcctaccacaggttactcccttccactggctgccaccctcgctgacgccttggctgccgaggctgacttctctcccgaagcactcgctcctcgtattcaccgatttgcccaggctgcctggcgaaagcaaggctttttcagaatgttgaatcgaatgctgtttcttgctgccgagggagatcgaagatggcgagtcatgcagcgtttctacggtctgcccgagggcttgattgcccgattctatgctggacgactcacacttgccgacagagctcggattctcagcggaaagcctcccgttcctgtgctggctgccctccaggccatccttactcatccttctggtcgaagagcttcacgataa

SEQ ID NO: 18 - protein, 387 aa, Enterobacteriaceae sp.
MTSQYDLLLLGAGLANGLLALRLKALQPQLRVLVLDAHAHAGGNHTWCFHEEDLSAAQHQWIAPLVAHRWPHYEVRFPALTRQLNSGYFCVTSARFDEVLRATLGDALRLNQTVASSGPDHVQLASGEVLRARAVIDGRGYQPDAALQIGFQSFVGQEWRLSQPHQLEGPILMDAAVDQQGGYRFVYTLPLSPTRLLIEDTHYINDASLATAQARQNICDYATRQGWQLETLLREERGALPITLAGDFDRFWHHRAPCVGLRAGLFHPTTGYSLPLAATLADALAAEADFSPEALAPRIHRFAQAAWRKQGFFRMLNRMLFLAAEGDRRWRVMQRFYGLPEGLIARFYAGRLTLADRARILSGKPPVPVLAALQAILTHPSGRRASR

SEQ ID NO: 19 - DNA, 980 bp, artificial sequence
  synthetic construct
cgatgcgtatctgtgggacatgtggtcgttgcgccattatgtaagcagcgtgtactcctctgactgtttaaaccatatggtttgctccatctcaccctcatcgttttcattgttcacaggcggccacaaaaaaactgtcttctctccttctctcttcgccttagtctactcggaccagttttagtttagcttggcgccactggataaatgagacctcaggccttgtgatgaggaggtcacttatgaagcatgttaggaggtgcttgtatggatagagaagcacccaaaataataagaataataataaaacagggggcgttgtcatttcatatcgtgttttcaccatcaatacacctccaaacaatgcccttcatgtggccagccccaatattgtcctgtagttcaactctatgcagctcgtatcttattgagcaagtaaaactctgtcagccgatattgcccgacccgcgacaagggtcaacaaggtggtgtaaggccttcgcagaagtcaaaactgtgccaaacaaacatctagagtctctttggtgtttctcgcatatatttwatcggctgtcttacgtatttgcgcctcggtaccggactaatttcggatcatccccaatacgctttttcttcgcagctgtcaacagtgtccatgatctatccacctaaatgggtcatatgaggcgtataatttcgtggtgctgataataattcccatatatttgacacaaaacttccccccctagacatacatctcacaatctcacttcttgtgcttctgtcacacatctcctccagctgacttcaactcacacctctgccccagttggtctacagcggtataaggtttctccgcatagaggtgcaccactcctcccgatacttgtttgtgtgacttgtgggtcacgacatatatatctacacacattgcgccaccctttggttcttccagcacaacaaaaacacgacacgctaac

SEQ ID NO: 20 - DNA, 339 bp, artificial sequence
  synthetic construct
attgatgattggaaacacacacatgggttatatctaggtgagagttagttggacagttatatattaaatcagctatgccaacggtaacttcattcatgtcaacgaggaaccagtgactgcaagtaatatagaatttgaccaccttgccattctcttgcactcctttactatatctcatttatttcttatatacaaatcacttcttcttcccagcatcgagctcggaaacctcatgagcaataacatcgtggatctcgtcaatagagggctttttggactccttgctgttggccaccttgtccttgctgtttaaactggctcattctgtttcaacgcctt

SEQ ID NO: 21 - DNA, 1335 bp, Chlamydomonas reinhardtii
atgggtcccggcatccagcctacctccgctcgaccctgttctcgaaccaagcactcccgattcgccctgctcgctgccgctcttactgctcgacgggtcaagcagttcaccaagcagtttcgatctcgacggatggccgaggacattctcaagctctggcaacgacagtaccaccttcctcgagaggattccgacaaacgaactctcagagaacgagtgcatctgtaccgtcctcccagatcggacctcggaggtatcgctgttgccgttaccgtcattgccttgtgggcaacactcttcgtgtacggactgtggttcgtcaagcttccctgggctctcaaggttggcgagacagccacttcctgggccaccatcgctgccgtgttctttagcctggagttcctctacaccggtctgttcattaccactcacgatgccatgcacggaaccattgcacttcgaaacagacgactcaacgactttctgggtcagcttgctatctctctgtacgcctggttcgactattccgttcttcatcgaaagcactgggagcatcacaaccataccggagagcctcgagtcgatcccgactttcaccgaggcaatcccaacctggccgtgtggtttgctcagttcatggtttcgtacatgactctttcccagtttctcaagattgccgtctggtccaacctgctccttctggctggagcacctcttgccaaccagctgctcttcatgaccgctgcacccatcctgagcgcttttcgacttttctactatggtacctacgttccacatcaccccgagaagggacacactggtgcgatgccctggcaagtctctcgaacaagctctgcctcccgactgcagtcgtttctcacctgctaccacttcgacttgcactgggagcatcacagatggccttacgcaccctggtgggagctgcccaagtgtcgacagattgcccgaggagctgcccttgctccaggtcccttgcctgtgccagctgccgcagctgccacagctgccactgcagctgccgcagccgctgccactggctctcctgctcccgcatcccgagctggttctgcttcctctgcctcggctgcagcttctggtttcggatctggccactccggatctgtcgctgcccaacccctgtcttccttgcctctgctctccgaaggcgtcaaaggtctggtcgagggtgctatggagctcgttgctggaggctcctcttcgggtggaggcggagagggtggcaagccaggtgctggcgaacacggactgctccagcgtcaacgacagctggcacccgttggagtcatggcttaa

SEQ ID NO: 22 - protein, 444 aa, Chlamydomonas reinhardtii
MGPGIQPTSARPCSRTKHSRFALLAAALTARRVKQFTKQFRSRRMAEDILKLWQRQYHLPREDSDKRTLRERVHLYRPPRSDLGGIAVAVTVIALWATLFVYGLWFVKLPWALKVGETATSWATIAAVFFSLEFLYTGLFITTHDAMHGTIALRNRRLNDFLGQLAISLYAWFDYSVLHRKHWEHHNHTGEPRVDPDFHRGNPNLAVWFAQFMVSYMTLSQFLKIAVWSNLLLLAGAPLANQLLFMTAAPILSAFRLFYYGTYVPHHPEKGHTGAMPWQVSRTSSASRLQSFLTCYHFDLHWEHHRWPYAPWWELPKCRQIARGAALAPGPLPVPAAAAATAATAAAAAAATGSPAPASRAGSASSASAAASGFGSGHSGSVAAQPLSSLPLLSEGVKGLVEGAMELVAGGSSSGGGGEGGKPGAGEHGLLQRQRQLAPVGVMA

SEQ ID NO: 23 - DNA, 988 bp, artificial sequence
  synthetic construct
gatctactatagaggaacaattgccccggagaagacggccaggccgcctagatgacaaattcaacaactcacagctgactttctgccattgccactaggggggggcctttttatatggccaagccaagctctccacgtcggttgggctgcacccaacaataaatgggtagggttgcaccaacaaagggatgggatggggggtagaagatacgaggataacggggctcaatggcacaaataagaacgaatactgccattaagactcgtgatccagcgactgacaccattgcatcatctaagggcctcaaaactacctcggaactgctgcgctgatctggacaccacagaggttccgagcactttaggttgcaccaaatgtcccaccaggtgcaggcagaaaacgctggaacagcgtgtacagtttgtcttaacaaaaagtgagggcgctgaggtcgagcagggtggtgtgacttgttatagcctttagagctgcgaaagcgcgtatggatttggctcatcaggccagattgagggtctgtggacacatgtcatgttagtgtacttcaatcgccccctggatatagccccgacaataggccgtggcctcatttttttgccttccgcacatttccattgctcggtacccacaccttgcttctcctgcacttgccaaccttaatactggtttacattgaccaacatcttacaagcggggggcttgtctagggtatatataaacagtggctctcccaatcggttgccagtctcttttttcctttctttccccacagattcgaaatctaaactacacatcacacaatgcctgttactgacgtccttaagcgaaagtccggtgtcatcgtcggcgacgatgtccgagccgtgagtatccacgacaagatcagtgtcgagacgacgcgttttgtgtaatgacacaatccgaaagtcgctagcaacacacactctctacacaaactaacccagctct

SEQ ID NO: 24 - DNA, 322 bp, artificial sequence; identical to SEQ ID NO: 8
  synthetic construct

SEQ ID NO: 25 - DNA, 489 bp, Brevundimonas vesicularis
atggcctcctggcccaccatgatcctcctgttccttgcaactttcctcggcatggaggtctttgcctgggctatgcaccgatacgtgatgcacggactgctctggacctggcaccgatctcatcatgaaccccacgacgatgtcttggagcgaaacgacctgtttgccgttgtcttcgctgcacctgccatcattctcgttgctcttggtctgcacttgtggccctggatgcttcccatcggactcggtgtcactgcctacggtctggtgtacttctttttccacgatggtcttgtccatcgtcgatttcctaccggaatcgctggcagatctgccttctggacacgacgtattcaggctcacagactgcatcacgccgttcgaacccgagagggctgtgtcagcttcggttttctctgggttcgatccgctcgagctctcaaggccgagctttcgcagaagcgaggctcttcctcgaacggagcttaa

SEQ ID NO: 26 - protein, 161 aa, Brevundimonas vesicularis
MSWPTMILLFLATFLGMEVFAWAMHRYVMHGLLWTWHRSHHEPHDDVLERNDLFAVVFAAPAIILVALGLHLWPWMLPIGLGVTAYGLVYFFFHDGLVHRRFPTGIAGRSAFWTRRIQAHRLHHAVRTREGCVSFGFLWVRSARALKAELSQKRGSSSNGA

SEQ ID NO: 27 - DNA, 904 bp, artificial sequence
  synthetic construct
ggaagccggtagaaccgggctgcttgtgcttggagatggaagccggtagaaccgggctgcttggggggatttggggccgctgggctccaaagaggggtaggcatttcgttggggttacgtaattgcggcatttgggtcctgcgcgcatgtcccattggtcagaattagtccggataggagacttatcagccaatcacagcgccggatccacctgtaggttgggttgggtgggagcacccctccacagagtagagtcaaacagcagcagcaacatgatagttgggggtgtgcgtgttaaaggaaaaaaaagaagcttgggttatattcccgctctatttagaggttgcgggatagacgccgacggagggcaatggcgctatggaaccttgcggatatccatacgccgcggcggactgcgtccgaaccagctccagcagcgttttttccgggccattgagccgactgcgaccccgccaacgtgtcttggcccacgcactcatgtcatgttggtgttgggaggccactttttaagtagcacaaggcacctagctcgcagcaaggtgtccgaaccaaagaagcggctgcagtggtgcaaacggggcggaaacggcgggaaaaagccacgggggcacgaattgaggcacgccctcgaatttgagacgagtcacggccccattcgcccgcgcaatggctcgccaacgcccggtcttttgcaccacatcaggttaccccaagccaaacctttgtgttaaaaagcttaacatattataccgaacgtaggtttgggcgggcttgctccgtctgtccaaggcaacatttatataagggtctgcatcgccggctcaattgaatcttttttcttcttctcttctctatattcattcttgaattaaacacacatcaa

SEQ ID NO: 28 - DNA, 307 bp, artificial sequence
  synthetic construct
attgatgattggaaacacacacatgggttatatctaggtgagagttagttggacagttatatattaaatcagctatgccaacggtaacttcattcatgtcaacgaggaaccagtgactgcaagtaatatagaatttgaccaccttgccattctcttgcactcctttactatatctcatttatttcttatatacaaatcacttcttcttcccagcatcgagctcggaaacctcatgagcaataacatcgtggatctcgtcaatagagggctttttggactccttgctgttggccaccttgtccttgct

SEQ ID NO: 29 - DNA, 891 bp, Arabidopsis thaliana
atggcctccttctcttcctcgtccaccgactttcgactgcgactccccaagtctctgtccggattctctccctcccttcgattcaagcgattctcggtctgctacgtcgtggaggaaagacgacagaactctcctatcgagaacgacgagcgacccgagtccaccagctctaccaacgctatcgacgccgagtacctggctctccgacttgccgagaagctggaacggaagaaatccgagcgatctacttacctcattgctgccatgctgtcctcgtttggcatcaccagcatggccgttatggctgtctattaccgattctcctggcagatggaaggaggcgagatttcgatgctggagatgttcggtacctttgccctctccgttggtgcagctgtcggcatggagttctgggctcgatgggcacatcgtgccttgtggcacgcgtcgctctggaacatgcacgagtctcatcacaagcctcgtgaaggtcccttcgagctcaacgacgtgtttgccattgtcaatgccggacctgcaatcggtctgctctcctacggctttttcaacaagggccttgttccaggactgtgtttcggtgctggactcggcatcaccgtgtttggcattgcctacatgtttgtccacgatggactggtgcacaagcgatttcctgtcggtcccattgccgatgttccctaccttcggaaggtcgctgccgcacatcagttgcaccataccgacaagttcaacggtgttccctacggactgtttcttggtcccaaggagctcgaagaggtcggaggcaacgaagagctcgacaaggagatctccagacgaatcaagtcttacaagaaagcttccggttcgggatcttccagctcttcgtaa

SEQ ID NO: 30 - protein, 310 aa, Arabidopsis thaliana
MAAGLSTAVTFKPLHRSFSSSSTDFRLRLPKSLSGFSPSLRFKRFSVCYVVEERRQNSPIENDERPESTSSTNAIDAEYLALRLAEKLERKKSERSTYLIAAMLSSFGITSMAVMAVYYRFSWQMEGGEISMLEMFGTFALSVGAAVGMEFWARWAHRALWHASLWNMHESHHKPREGPFELNDVFAIVNAGPAIGLLSYGFFNKGLVPGLCFGAGLGITVFGIAYMFVHDGLVHKRFPVGPIADVPYLRKVAAAHQLHHTDKFNGVPYGLFLGPKELEEVGGNEELDKEISRRIKSYKKASGSGSSSSS

SEQ ID NO: 31 - DNA, 904 bp, artificial sequence; identical to SEQ ID NO: 27
  synthetic construct

SEQ ID NO: 32 - DNA, 307 bp, artificial sequence; identical to SEQ ID NO: 28
  synthetic construct

SEQ ID NO: 33 - DNA, 36 bp, artificial sequence
  synthetic construct
actttaattaacgatgcgtatctgtgggacatgtgg

SEQ ID NO: 34 - DNA, 34 bp, artificial sequence
  synthetic construct
tcaccatgggttagcgtgtcgtgtttttgttgtg

SEQ ID NO: 35 - DNA, 36 bp, artificial sequence
  synthetic construct
actgcggccgcattgatgattggaaacacacacatg

SEQ ID NO: 36 - DNA, 32 bp, artificial sequence
  synthetic construct
actgaattcaaggcgttgaaacagaatgagcc

SEQ ID NO: 37 - DNA, 10539 bp, artificial sequence
  synthetic construct
atcgatggaagccggtagaaccgggctgcttgtgcttggagatggaagccggtagaaccgggctgcttggggggatttggggccgctgggctccaaagaggggtaggcatttcgttggggttacgtaattgcggcatttgggtcctgcgcgcatgtcccattggtcagaattagtccggataggagacttatcagccaatcacagcgccggatccacctgtaggttgggttgggtgggagcacccctccacagagtagagtcaaacagcagcagcaacatgatagttgggggtgtgcgtgttaaaggaaaaaaaagaagcttgggttatattcccgctctatttagaggttgcgggatagacgccgacggagggcaatggcgctatggaaccttgcggatatccatacgccgcggcggactgcgtccgaaccagctccagcagcgttttttccgggccattgagccgactgcgaccccgccaacgtgtcttggcccacgcactcatgtcatgttggtgttgggaggccactttttaagtagcacaaggcacctagctcgcagcaaggtgtccgaaccaaagaagcggctgcagtggtgcaaacggggcggaaacggcgggaaaaagccacgggggcacgaattgaggcacgccctcgaatttgagacgagtcacggccccattcgcccgcgcaatggctcgccaacgcccggtcttttgcaccacatcaggttaccccaagccaaacctttgtgttaaaaagcttaacatattataccgaacgtaggtttgggcgggcttgctccgtctgtccaaggcaacatttatataagggtctgcatcgccggctcaattgaatcttttttcttcttctcttctctatattcattcttgaattaaacacacatcaaccatggcctcctggcccaccatgatcctcctgttccttgcaactttcctcggcatggaggtctttgcctgggctatgcaccgatacgtgatgcacggactgctctggacctggcaccgatctcatcatgaaccccacgacgatgtcttggagcgaaacgacctgtttgccgttgtcttcgctgcacctgccatcattctcgttgctcttggtctgcacttgtggccctggatgcttcccatcggactcggtgtcactgcctacggtctggtgtacttctttttccacgatggtcttgtccatcgtcgatttcctaccggaatcgctggcagatctgccttctggacacgacgtattcaggctcacagactgcatcacgccgttcgaacccgagagggctgtgtcagcttcggttttctctgggttcgatccgctcgagctctcaaggccgagctttcgcagaagcgaggctcttcctcgaacggagcttaagcggccgcattgatgattggaaacacacacatgggttatatctaggtgagagttagttggacagttatatattaaatcagctatgccaacggtaacttcattcatgtcaacgaggaaccagtgactgcaagtaatatagaatttgaccaccttgccattctcttgcactcctttactatatctcatttatttcttatatacaaatcacttcttcttcccagcatcgagctcggaaacctcatgagcaataacatcgtggatctcgtcaatagagggctttttggactccttgctgttggccaccttgtccttgctgtttaaacaccactaaaaccccacaaaatatatcttaccgaatatacagatctactatagaggaacaattgccccggagaagacggccaggccgcctagatgacaaattcaacaactcacagctgactttctgccattgccactaggggggggcctttttatatggccaagccaagctctccacgtcggttgggctgcacccaacaataaatgggtagggttgcaccaacaaagggatgggatggggggtagaagatacgaggataacggggctcaatggcacaaataagaacgaatactgccattaagactcgtgatccagcgactgacaccattgcatcatctaagggcctcaaaactacctcggaactgctgcgctgatctggacaccacagaggttccgagcactttaggttgcaccaaatgtcccaccaggtgcaggcagaaaacgctggaacagcgtgtacagtttgtcttaacaaaaagtgagggcgctgaggtcgagcagggtggtgtgacttgttatagcctttagagctgcgaaagcgcgtatggatttggctcatcaggccagattgagggtctgtggacacatgtcatgttagtgtacttcaatcgccccctggatatagccccgacaataggccgtggcctcatttttttgccttccgcacatttccattgctcggtacccacaccttgcttctcctgcacttgccaaccttaatactggtttacattgaccaacatcttacaagcggggggcttgtctagggtatatataaacagtggctctcccaatcggttgccagtctcttttttcctttctttccccacagattcgaaatctaaactacacatcacacaatgcctgttactgacgtccttaagcgaaagtccggtgtcatcgtcggcgacgatgtccgagccgtgagtatccacgacaagatcagtgtcgagacgacgcgttttgtgtaatgacacaatccgaaagtcgctagcaacacacactctctacacaaactaacccagctctccatgggtcccggcatccagcctacctccgctcgaccctgttctcgaaccaagcactcccgattcgccctgctcgctgccgctcttactgctcgacgggtcaagcagttcaccaagcagtttcgatctcgacggatggccgaggacattctcaagctctggcaacgacagtaccaccttcctcgagaggattccgacaaacgaactctcagagaacgagtgcatctgtaccgtcctcccagatcggacctcggaggtatcgctgttgccgttaccgtcattgccttgtgggcaacactcttcgtgtacggactgtggttcgtcaagcttccctgggctctcaaggttggcgagacagccacttcctgggccaccatcgctgccgtgttctttagcctggagttcctctacaccggtctgttcattaccactcacgatgccatgcacggaaccattgcacttcgaaacagacgactcaacgactttctgggtcagcttgctatctctctgtacgcctggttcgactattccgttcttcatcgaaagcactgggagcatcacaaccataccggagagcctcgagtcgatcccgactttcaccgaggcaatcccaacctggccgtgtggtttgctcagttcatggtttcgtacatgactctttcccagtttctcaagattgccgtctggtccaacctgctccttctggctggagcacctcttgccaaccagctgctcttcatgaccgctgcacccatcctgagcgcttttcgacttttctactatggtacctacgttccacatcaccccgagaagggacacactggtgcgatgccctggcaagtctctcgaacaagctctgcctcccgactgcagtcgtttctcacctgctaccacttcgacttgcactgggagcatcacagatggccttacgcaccctggtgggagctgcccaagtgtcgacagattgcccgaggagctgcccttgctccaggtcccttgcctgtgccagctgccgcagctgccacagctgccactgcagctgccgcagccgctgccactggctctcctgctcccgcatcccgagctggttctgcttcctctgcctcggctgcagcttctggtttcggatctggccactccggatctgtcgctgcccaacccctgtcttccttgcctctgctctccgaaggcgtcaaaggtctggtcgagggtgctatggagctcgttgctggaggctcctcttcgggtggaggcggagagggtggcaagccaggtgctggcgaacacggactgctccagcgtcaacgacagctggcacccgttggagtcatggcttaagcggccgcatgagaagataaatatataaatacattgagatattaaatgcgctagattagagagcctcatactgctcggagagaagccaagacgagtactcaaaggggattacaccatccatatccacagacacaagctggggaaaggttctatatacactttccggaataccgtagtttccgatgttatcaatgggggcagccaggatttcaggcacttcggtgtctcggggtgaaatggcgttcttggcctccatcaagtcgtaccatgtcttcatttgcctgtcaaagtaaaacagaagcagatgaagaatgaacttgaagtgaaggaatttaaatgtaacgaaactgaaatttgaccagatattgtgtccgcggtggagctccagcttttgttccctttagtgagggttaatttcgagcttggcgtaatcatggtcatagctgtttcctgtgtgaaattgttatccgctcacaagcttccacacaacgtacgccaccattctgtctgccgccatgatgctcaagttctctcttaacatgaagcccgccggtgacgctgttgaggctgccgtcaaggagtccgtcgaggctggtatcactaccgccgatatcggaggctcttcctccacctccgaggtcggagacttgttgccaacaaggtcaaggagctgctcaagaaggagtaagtcgtttctacgacgcattgatggaaggagcaaactgacgcgcctgcgggttggtctaccggcagggtccgctagtgtataagactctataaaaagggccctgccctgctaatgaaatgatgatttataatttaccggtgtagcaaccttgactagaagaagcagattgggtgtgtttgtagtggaggacagtggtacgttttggaaacagtcttcttgaaagtgtcttgtctacagtatattcactcataacctcaatagccaagggtgtagtcggtttattaaaggaagggagttgtggctgatgtggatagatatctttaagctggcgactgcacccaacgagtgtggtggtagcttgttactgtatattcggtaagatatattttgtggggttttagtggtgtttggtaggttagtgcttggtatatgagttgtaggcatgacaatttggaaaggggtggactttgggaatattgtgggatttcaataccttagtttgtacagggtaattgttacaaatgatacaaagaactgtatttcttttcatttgttttaattggttgtatatcaagtccgttagacgagctcagtgggcgcgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgctatcggcatcgacaaggtttgggtccctagccgataccgcactacctgagtcacaatcttcggaggtttagtcttccacatagcacgggcaaaagtgcgtatatatacaagagcgtttgccagccacagattttcactccacacaccacatcacacatacaaccacacacatccacaatggaacccgaaactaagaagaccaagactgactccaagaagattgttcttctcggcggcgacttctgtggccccgaggtgattgccgaggccgtcaaggtgctcaagtctgttgctgaggcctccggcaccgagtttgtgtttgaggaccgactcattggaggagctgccattgagaaggagggcgagcccatcaccgacgctactctcgacatctgccgaaaggctgactctattatgctcggtgctgtcggaggcgctgccaacaccgtatggaccactcccgacggacgaaccgacgtgcgacccgagcagggtctcctcaagctgcgaaaggacctgaacctgtacgccaacctgcgaccctgccagctgctgtcgcccaagctcgccgatctctcccccatccgaaacgttgagggcaccgacttcatcattgtccgagagctcgtcggaggtatctactttggagagcgaaaggaggatgacggatctggcgtcgcttccgacaccgagacctactccgttaattaactttggccggaattcctttacctgcaggataacttcgtataatgtatgctatacgaagttatgatctctctcttgagcttttccataacaagttcttctgcctccaggaagtccatgggtggtttgatcatggttttggtgtagtggtagtgcagtggtggtattgtgactggggatgtagttgagaataagtcatacacaagtcagctttcttcgagcctcatataagtataagtagttcaacgtattagcactgtacccagcatctccgtatcgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaattacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggactaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtgacaccggtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggttttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgtcgcctttgccgacaaaaatgtgatcatgatgaaagccagcaatgacgttgcagctgatattgttgtcggccaaccgcgccgaaaacgcagctgtcagacccacagcctccaacgaagaatgtatcgtcaaagtgatccaagcacactcatagttggagtcgtactccaaaggcggcaatgacgagtcagacagatactcgtcgacgcgataacttcgtataatgtatgctatacgaagttatcgtacgatagttagtagacaaca

SEQ ID NO: 38 - DNA, 10941 bp, artificial sequence
  synthetic construct
aatgggggcagccaggatttcaggcacttcggtgtctcggggtgaaatggcgttcttggcctccatcaagtcgtaccatgtcttcatttgcctgtcaaagtaaaacagaagcagatgaagaatgaacttgaagtgaaggaatttaaatgtaacgaaactgaaatttgaccagatattgtgtccgcggtggagctccagcttttgttccctttagtgagggttaatttcgagcttggcgtaatcatggtcatagctgtttcctgtgtgaaattgttatccgctcacaagcttccacacaacgtacgccaccattctgtctgccgccatgatgctcaagttctctcttaacatgaagcccgccggtgacgctgttgaggctgccgtcaaggagtccgtcgaggctggtatcactaccgccgatatcggaggctcttcctccacctccgaggtcggagacttgttgccaacaaggtcaaggagctgctcaagaaggagtaagtcgtttctacgacgcattgatggaaggagcaaactgacgcgcctgcgggttggtctaccggcagggtccgctagtgtataagactctataaaaagggccctgccctgctaatgaaatgatgatttataatttaccggtgtagcaaccttgactagaagaagcagattgggtgtgtttgtagtggaggacagtggtacgttttggaaacagtcttcttgaaagtgtcttgtctacagtatattcactcataacctcaatagccaagggtgtagtcggtttattaaaggaagggagttgtggctgatgtggatagatatctttaagctggcgactgcacccaacgagtgtggtggtagcttgttactgtatattcggtaagatatattttgtggggttttagtggtgtttggtaggttagtgcttggtatatgagttgtaggcatgacaatttggaaaggggtggactttgggaatattgtgggatttcaataccttagtttgtacagggtaattgttacaaatgatacaaagaactgtatttcttttcatttgttttaattggttgtatatcaagtccgttagacgagctcagtgggcgcgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgctatcggcatcgacaaggtttgggtccctagccgataccgcactacctgagtcacaatcttcggaggtttagtcttccacatagcacgggcaaaagtgcgtatatatacaagagcgtttgccagccacagattttcactccacacaccacatcacacatacaaccacacacatccacaatggaacccgaaactaagaagaccaagactgactccaagaagattgttcttctcggcggcgacttctgtggccccgaggtgattgccgaggccgtcaaggtgctcaagtctgttgctgaggcctccggcaccgagtttgtgtttgaggaccgactcattggaggagctgccattgagaaggagggcgagcccatcaccgacgctactctcgacatctgccgaaaggctgactctattatgctcggtgctgtcggaggcgctgccaacaccgtatggaccactcccgacggacgaaccgacgtgcgacccgagcagggtctcctcaagctgcgaaaggacctgaacctgtacgccaacctgcgaccctgccagctgctgtcgcccaagctcgccgatctctcccccatccgaaacgttgagggcaccgacttcatcattgtccgagagctcgtcggaggtatctactttggagagcgaaaggaggatgacggatctggcgtcgcttccgacaccgagacctactccgttaattaactttggccggaattcctttacctgcaggataacttcgtataatgtatgctatacgaagttatgatctctctcttgagcttttccataacaagttcttctgcctccaggaagtccatgggtggtttgatcatggttttggtgtagtggtagtgcagtggtggtattgtgactggggatgtagttgagaataagtcatacacaagtcagctttcttcgagcctcatataagtataagtagttcaacgtattagcactgtacccagcatctccgtatcgagaaacacaacaacatgccccattggacagatcatgcggatacacaggttgtgcagtatcatacatactcgatcagacaggtcgtctgaccatcatacaagctgaacaagcgctccatacttgcacgctctctatatacacagttaaattacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcgtctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggactaggaactccttgtactgggagttctcgtagtcagagacgtcctccttcttctgttcagagacagtttcctcggcaccagctcgcaggccagcaatgattccggttccgggtacaccgtgggcgttggtgatatcggaccactcggcgattcggtgacaccggtactggtgcttgacagtgttgccaatatctgcgaactttctgtcctcgaacaggaagaaaccgtgcttaagagcaagttccttgagggggagcacagtgccggcgtaggtgaagtcgtcaatgatgtcgatatgggttttgatcatgcacacataaggtccgaccttatcggcaagctcaatgagctccttggtggtggtaacatccagagaagcacacaggttggttttcttggctgccacgagcttgagcactcgagcggcaaaggcggacttgtggacgttagctcgagcttcgtaggagggcattttggtggtgaagaggagactgaaataaatttagtctgcagaactttttatcggaaccttatctggggcagtgaagtatatgttatggtaatagttacgagttagttgaacttatagatagactggactatacggctatcggtccaaattagaaagaacgtcaatggctctctgggcgtcgcctttgccgacaaaaatgtgatcatgatgaaagccagcaatgacgttgcagctgatattgttgtcggccaaccgcgccgaaaacgcagctgtcagacccacagcctccaacgaagaatgtatcgtcaaagtgatccaagcacactcatagttggagtcgtactccaaaggcggcaatgacgagtcagacagatactcgtcgacgcgataacttcgtataatgtatgctatacgaagttatcgtacgatagttagtagacaacaatcgatggaagccggtagaaccgggctgcttgtgcttggagatggaagccggtagaaccgggctgcttggggggatttggggccgctgggctccaaagaggggtaggcatttcgttggggttacgtaattgcggcatttgggtcctgcgcgcatgtcccattggtcagaattagtccggataggagacttatcagccaatcacagcgccggatccacctgtaggttgggttgggtgggagcacccctccacagagtagagtcaaacagcagcagcaacatgatagttgggggtgtgcgtgttaaaggaaaaaaaagaagcttgggttatattcccgctctatttagaggttgcgggatagacgccgacggagggcaatggcgctatggaaccttgcggatatccatacgccgcggcggactgcgtccgaaccagctccagcagcgttttttccgggccattgagccgactgcgaccccgccaacgtgtcttggcccacgcactcatgtcatgttggtgttgggaggccactttttaagtagcacaaggcacctagctcgcagcaaggtgtccgaaccaaagaagcggctgcagtggtgcaaacggggcggaaacggcgggaaaaagccacgggggcacgaattgaggcacgccctcgaatttgagacgagtcacggccccattcgcccgcgcaatggctcgccaacgcccggtcttttgcaccacatcaggttaccccaagccaaacctttgtgttaaaaagcttaacatattataccgaacgtaggtttgggcgggcttgctccgtctgtccaaggcaacatttatataagggtctgcatcgccggctcaattgaatcttttttcttcttctcttctctatattcattcttgaattaaacacacatcaaccatggcctccttctcttcctcgtccaccgactttcgactgcgactccccaagtctctgtccggattctctccctcccttcgattcaagcgattctcggtctgctacgtcgtggaggaaagacgacagaactctcctatcgagaacgacgagcgacccgagtccaccagctctaccaacgctatcgacgccgagtacctggctctccgacttgccgagaagctggaacggaagaaatccgagcgatctacttacctcattgctgccatgctgtcctcgtttggcatcaccagcatggccgttatggctgtctattaccgattctcctggcagatggaaggaggcgagatttcgatgctggagatgttcggtacctttgccctctccgttggtgcagctgtcggcatggagttctgggctcgatgggcacatcgtgccttgtggcacgcgtcgctctggaacatgcacgagtctcatcacaagcctcgtgaaggtcccttcgagctcaacgacgtgtttgccattgtcaatgccggacctgcaatcggtctgctctcctacggctttttcaacaagggccttgttccaggactgtgtttcggtgctggactcggcatcaccgtgtttggcattgcctacatgtttgtccacgatggactggtgcacaagcgatttcctgtcggtcccattgccgatgttccctaccttcggaaggtcgctgccgcacatcagttgcaccataccgacaagttcaacggtgttccctacggactgtttcttggtcccaaggagctcgaagaggtcggaggcaacgaagagctcgacaaggagatctccagacgaatcaagtcttacaagaaagcttccggttcgggatcttccagctcttcgtaagcggccgcattgatgattggaaacacacacatgggttatatctaggtgagagttagttggacagttatatattaaatcagctatgccaacggtaacttcattcatgtcaacgaggaaccagtgactgcaagtaatatagaatttgaccaccttgccattctcttgcactcctttactatatctcatttatttcttatatacaaatcacttcttcttcccagcatcgagctcggaaacctcatgagcaataacatcgtggatctcgtcaatagagggctttttggactccttgctgttggccaccttgtccttgctgtttaaacaccactaaaaccccacaaaatatatcttaccgaatatacagatctactatagaggaacaattgccccggagaagacggccaggccgcctagatgacaaattcaacaactcacagctgactttctgccattgccactaggggggggcctttttatatggccaagccaagctctccacgtcggttgggctgcacccaacaataaatgggtagggttgcaccaacaaagggatgggatggggggtagaagatacgaggataacggggctcaatggcacaaataagaacgaatactgccattaagactcgtgatccagcgactgacaccattgcatcatctaagggcctcaaaactacctcggaactgctgcgctgatctggacaccacagaggttccgagcactttaggttgcaccaaatgtcccaccaggtgcaggcagaaaacgctggaacagcgtgtacagtttgtcttaacaaaaagtgagggcgctgaggtcgagcagggtggtgtgacttgttatagcctttagagctgcgaaagcgcgtatggatttggctcatcaggccagattgagggtctgtggacacatgtcatgttagtgtacttcaatcgccccctggatatagccccgacaataggccgtggcctcatttttttgccttccgcacatttccattgctcggtacccacaccttgcttctcctgcacttgccaaccttaatactggtttacattgaccaacatcttacaagcggggggcttgtctagggtatatataaacagtggctctcccaatcggttgccagtctcttttttcctttctttccccacagattcgaaatctaaactacacatcacacaatgcctgttactgacgtccttaagcgaaagtccggtgtcatcgtcggcgacgatgtccgagccgtgagtatccacgacaagatcagtgtcgagacgacgcgttttgtgtaatgacacaatccgaaagtcgctagcaacacacactctctacacaaactaacccagctctccatgggtcccggcatccagcctacctccgctcgaccctgttctcgaaccaagcactcccgattcgccctgctcgctgccgctcttactgctcgacgggtcaagcagttcaccaagcagtttcgatctcgacggatggccgaggacattctcaagctctggcaacgacagtaccaccttcctcgagaggattccgacaaacgaactctcagagaacgagtgcatctgtaccgtcctcccagatcggacctcggaggtatcgctgttgccgttaccgtcattgccttgtgggcaacactcttcgtgtacggactgtggttcgtcaagcttccctgggctctcaaggttggcgagacagccacttcctgggccaccatcgctgccgtgttctttagcctggagttcctctacaccggtctgttcattaccactcacgatgccatgcacggaaccattgcacttcgaaacagacgactcaacgactttctgggtcagcttgctatctctctgtacgcctggttcgactattccgttcttcatcgaaagcactgggagcatcacaaccataccggagagcctcgagtcgatcccgactttcaccgaggcaatcccaacctggccgtgtggtttgctcagttcatggtttcgtacatgactctttcccagtttctcaagattgccgtctggtccaacctgctccttctggctggagcacctcttgccaaccagctgctcttcatgaccgctgcacccatcctgagcgcttttcgacttttctactatggtacctacgttccacatcaccccgagaagggacacactggtgcgatgccctggcaagtctctcgaacaagctctgcctcccgactgcagtcgtttctcacctgctaccacttcgacttgcactgggagcatcacagatggccttacgcaccctggtgggagctgcccaagtgtcgacagattgcccgaggagctgcccttgctccaggtcccttgcctgtgccagctgccgcagctgccacagctgccactgcagctgccgcagccgctgccactggctctcctgctcccgcatcccgagctggttctgcttcctctgcctcggctgcagcttctggtttcggatctggccactccggatctgtcgctgcccaacccctgtcttccttgcctctgctctccgaaggcgtcaaaggtctggtcgagggtgctatggagctcgttgctggaggctcctcttcgggtggaggcggagagggtggcaagccaggtgctggcgaacacggactgctccagcgtcaacgacagctggcacccgttggagtcatggcttaagcggccgcatgagaagataaatatataaatacattgagatattaaatgcgctagattagagagcctcatactgctcggagagaagccaagacgagtactcaaaggggattacaccatccatatccacagacacaagctggggaaaggttctatatacactttccggaataccgtagtttccgatgttatc

(19) United States
(12) Patent Application Publication
(10) Pub. No.: US 2015/0167041 A1
(43) Pub. Date: Jun. 18, 2015

(54) ASTAXANTHIN PRODUCTION USING A RECOMBINANT MICROBIAL HOST CELL

(71) Applicant: E I DU PONT DE NEMOURS AND COMPANY, Wilmington, DE (US)

(72) Inventors: SUDIP KUMAR SAMANTA, Hyderabad (IN); Anirban Banerjee, West Bengal (IN); Ashish Paradkar, Hyderabad (IN); Qiong Cheng, Wilmington, DE (US); Pamela L Sharpe, Wilmington, DE (US); Quinn Qun Zhu, West Chester, PA (US)

(21) Appl. No.: 14/521,625

(22) Filed: Oct. 23, 2014

(30) Foreign Application Priority Data
Dec. 14, 2013 (IN) 3659/DEL/2013

(51) Int. Cl. C12P 23/00; A61K 31/122; A23K 1/16; C12N 15/81

(52) U.S. Cl. CPC C12P 23/00; C12N 15/81; A61K 31/122; A23K 1/1606

(57) ABSTRACT

A recombinant microbial host cell is provided capable of producing astaxanthin from β-carotene without a measurable concomitant accumulation of ketolated or hydroxylated intermediates such as adonixanthin, zeaxanthin, adonirubin, echinenone, 3-hydroxyechinenone, 3′-hydroxyechinenone, canthaxanthin, and β-cryptoxanthin. Specifically, a β-carotene producing microbial host cell was engineered to express two heterologous genes, a β-carotene ketolase from Chlamydomonas reinhardtii in combination with a carotenoid hydroxylase from Brevundimonas vesicularis or Arabidopsis thaliana.

### CROSS-REFERENCE TO RELATED APPLICATIONS

This application claims benefit of Indian Provisional Patent Application No. 3659/DEL/2013, filed Dec. 14, 2013, which is incorporated by reference herein in its entirety.

### FIELD OF THE INVENTION

This invention is in the field of biotechnology. More specifically, this invention pertains to a process of producing astaxanthin from β-carotene in a recombinant microbial host cell engineered to express a specified combination of a carotenoid ketolase and a carotenoid hydroxylase that facilitates production of astaxanthin without significant accumulation of ketolated or hydroxylated carotenoid intermediates.

### BACKGROUND OF THE INVENTION

Carotenoids (e.g., lycopene, β-carotene, zeaxanthin, canthaxanthin and astaxanthin) represent one of the most widely distributed and structurally diverse classes of natural pigments, producing pigment colors of light yellow to orange to deep red color. Eye-catching examples of carotenogenic tissues include carrots, tomatoes, red peppers, and the petals of daffodils and marigolds. Carotenoids are synthesized by all photosynthetic organisms, as well as some bacteria and fungi. These pigments have important functions in photosynthesis, nutrition, and protection against photooxidative damage; as such, they are used today in food ingredients/colors, animal feed ingredients, pharmaceuticals, cosmetics and as nutritional supplements.

Animals do not have the ability to synthesize carotenoids but must obtain these nutritionally important compounds through their dietary sources. Many animals exhibit an increase in tissue pigmentation when carotenoids are included in their diets, a characteristic often valued by consumers. For example, canthaxanthin and astaxanthin are commonly used in commercial aquaculture industries to pigment shrimp and salmonid fish. It has also been reported that astaxanthin may be a dietary requirement for the growth and survival of some salmonid species (Christiansen et al., Aquaculture Nutrition, 1:189-198 (1995)). Similarly, lutein, canthaxanthin and astaxanthin are commonly used as pigments in poultry feeds to increase the pigmentation of chicken skin and egg yolks.

Industrially, only a few carotenoids are used, despite the existence of more than 600 different carotenoids identified in nature. This is largely due to difficulties in production and high associated costs. For example, the predominant source of aquaculture pigments used in the market today are produced synthetically and are sold under such trade names as CAROPHYLL® Pink (astaxanthin; DSM Nutritional Products; Kaiseraugst, Switzerland); however, the cost of utilizing the synthetically produced pigments is quite high even though the amount of pigment incorporated into the fishmeal is typically less than 100 ppm.

Natural carotenoids can either be obtained by extraction of plant material or by microbial synthesis; but, only a few plants are widely used for commercial carotenoid production and the productivity of carotenoid synthesis in these plants is relatively low. Microbial production of carotenoids is a more attractive production route. Examples of carotenoid-producing microorganisms include: algae (Haematococcus pluvialis, sold under the tradename NATUROSE™ (Cyanotech Corp., Kailua-Kona, Hi.; Dunaliella sp.), yeast (Phaffia rhodozyma; also referred to as Xanthophyllomyces dendrorhous; Thraustochytrium sp.; Labyrinthula sp.; and Saccharomyces cerevisiae), and bacteria (Paracoccus marcusii, Bradyrhizobium, Rhodobacter sp., Brevibacterium, Escherichia coli and Methylomonas sp.).

Many of the genes involved in carotenoid biosynthesis have been heterologously expressed in a variety of host cells such as Escherichia coli, Candida utilis, Saccharomyces cerevisiae, Yarrowia lipolytica, and Methylomonas sp. U.S. Pat. No. 6,969,595 to Brzostowicz et al. describes carotenoid production in recombinant microbial host cell from single carbon substrates. U.S. Patent Appl. Pub. No. 2012-0142082A1 to Sharpe et al. discloses carotenoid production in a recombinant oleaginous yeast. The oleaginous yeast may be further modified to produce at least one ω-3 and/or ω-6 polyunsaturated fatty acid.

U.S. Pat. Nos. 7,851,199 and 8,288,149, and U.S. Patent Appl. Pub. No. 2013-0045504 to Baily et al. disclose an engineered oleaginous yeast to produce carotenoids, thereby resulting in a pigmented microbial product.

Recombinant microbial production of β-carotene has been demonstrated in a variety of host cells. However, converting β-carotene to astaxanthin requires expression of at least one gene encoding a carotenoid ketolase and expression of at least gene encoding a carotenoid hydroxylase. Enzymatic synthesis of astaxanthin from β-carotene typically produces a variety of possible “intermediates” such as β-cryptoxanthin, zeaxanthin, adonixanthin, 3-hydroxyechinenone, 3′-hydroxyechinenone, echinenone, canthaxanthin, and adonirubin. The carotenoid ketolase and/or carotenoid hydroxylase may not have significant specific activity towards one or more of these intermediates, often leading to the concomitant accumulation of one or more of the above intermediates and decreasing the production of astaxanthin. Separation of astaxanthin from one or more of these accumulated intermediates adds cost and may make recombinant microbial production less attractive. As such, engineering a recombinant microbial host cell capable of producing β-carotene to express a combination of at least one carotenoid ketolase and at least one carotenoid hydroxylase that does not result in the undesirable accumulation of an intermediate when producing astaxanthin is needed.

The problem to be solved therefore, is to provide a recombinant microbial host cell (capable of producing β-carotene either naturally or recombinantly) which expresses a combination of genes encoding at least one carotenoid ketolase and at least one carotenoid hydroxylase wherein the engineered strain does not accumulate a significant amount of an intermediate when producing astaxanthin.

### SUMMARY OF THE INVENTION

The stated problem has been solved by providing a recombinant microbial host cell capable of producing a significant amount of astaxanthin without a significant accumulation of a ketolated and/or hydroxylated carotenoid intermediate when converting β-carotene to astaxanthin.

In one embodiment, a recombinant microbial host cell is provided comprising:
a. a set of β-carotene biosynthesis pathway genes;
b. at least one expressible genetic construct encoding the 6-carotene ketolase from Chlamydomonas reinhardtii; and
c. at least one expressible genetic construct encoding a carotenoid hydroxylase selected from Brevundimonas sp., Arabidopsis thaliana or a combination thereof;
   wherein the recombinant microbial host cell produces astaxanthin from β-carotene and does not concomitantly accumulate a significant amount of any one of the following ketolated and/or hydroxylated carotenoid intermediates: adonixanthin, zeaxanthin, adonirubin, echinenone, 3-hydroxyechinenone, 3′-hydroxyechinenone, canthaxanthin or 6-cryptoxanthin; wherein the ratio of astaxanthin to any one of the ketolasted and/or hydroxylated carotenoid intermediates as measured by dry cell weight is at least 75:1, preferably at least 100:1, more preferably at least 125:1, and most preferably at least 150:1.

In another embodiment, a method to produce astaxanthin is provided comprising:

a. providing the present recombinant microbial host cell; and

b. growing the recombinant microbial host cell whereby astaxanthin is produced.

In another embodiment, a method to produce an animal feed comprising astaxanthin is provided comprising:

a. providing the astaxanthin produced by the present recombinant microbial host cell;

b. adding an effective amount of the astaxanthin to an animal feed whereby an animal feed comprising astaxanthin is produced.

In another embodiment, a method to pigment the muscle tissue of an animal is provided comprising:

a. providing the above animal feed comprising astaxanthin;

b. feeding an animal the animal feed comprising astaxanthin whereby the muscle tissue of the animal is pigmented by the astaxanthin present in the animal feed.

### BRIEF DESCRIPTION OF THE FIGURES, AND SEQUENCE DESCRIPTIONS

The invention can be more fully understood from the following figures, sequence descriptions, and the detailed description.

### BRIEF DESCRIPTION OF THE FIGURES

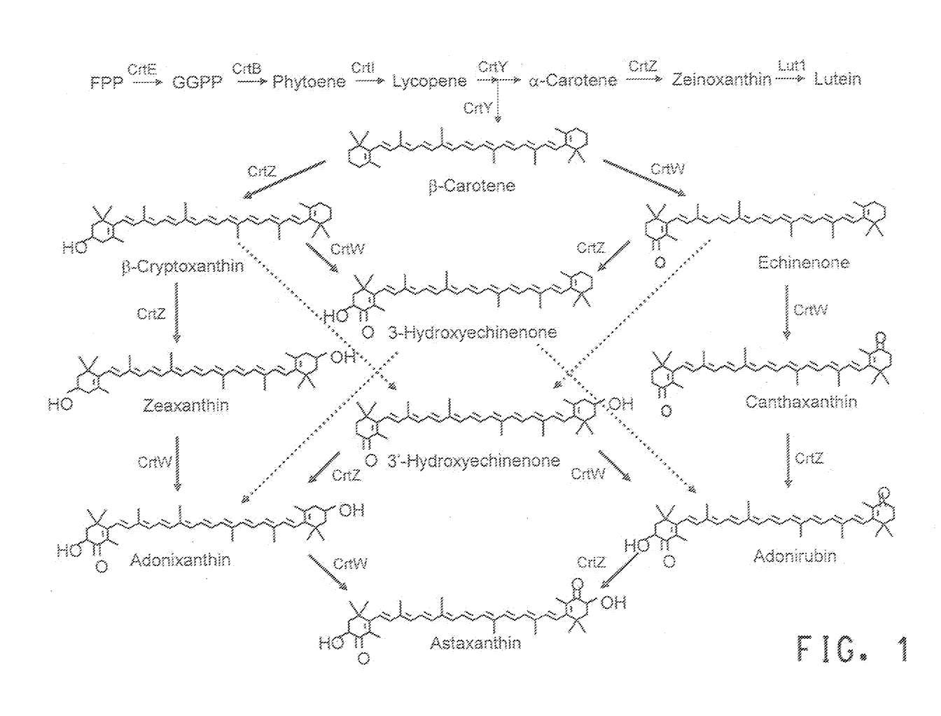

FIG. 1 illustrates the biosynthetic pathway from farnesyl pyrophosphate (FPP) to astaxanthin. The enzymes necessary to produce β-carotene (the β-carotene synthesis pathway genes) from FPP are CrtE, CrtB, CrtI, and CrtY. Production of astaxanthin from β-carotene requires a combination of at least one β-carotene ketolase (CrtW/CrtO/Bkt) and at least one carotenoid hydroxylase (CrtZ).

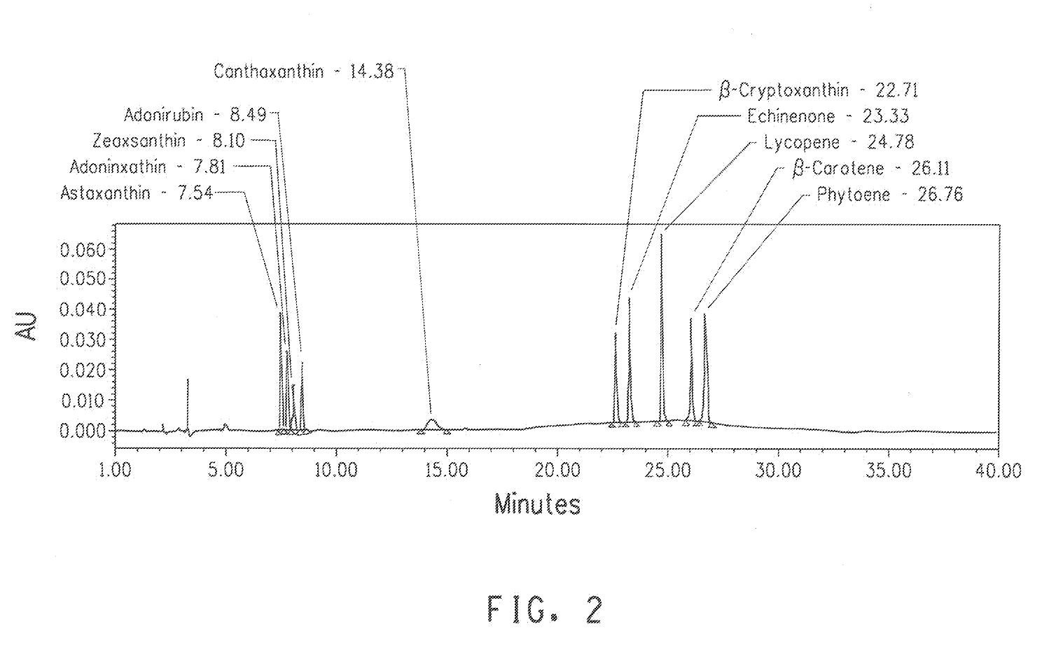

FIG. 2 illustrates a chromatogram showing separation of various carotenoid intermediates as standards.

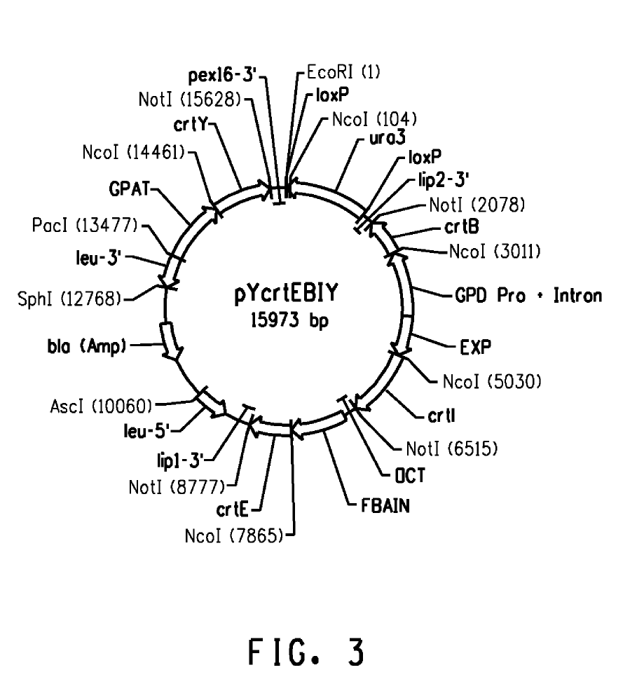

FIG. 3 is a plasmid map for pYcrtEBIY.

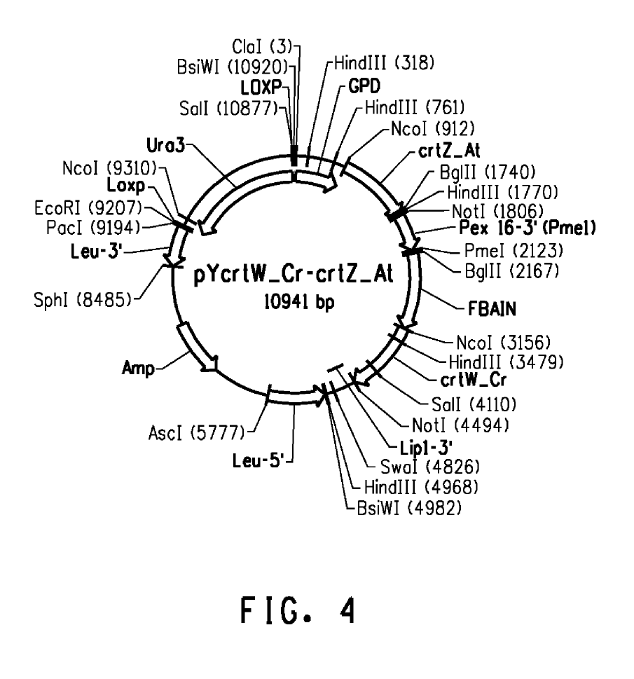

FIG. 4 is a plasmid map for pYcrtW_Cr-crtZ_At.

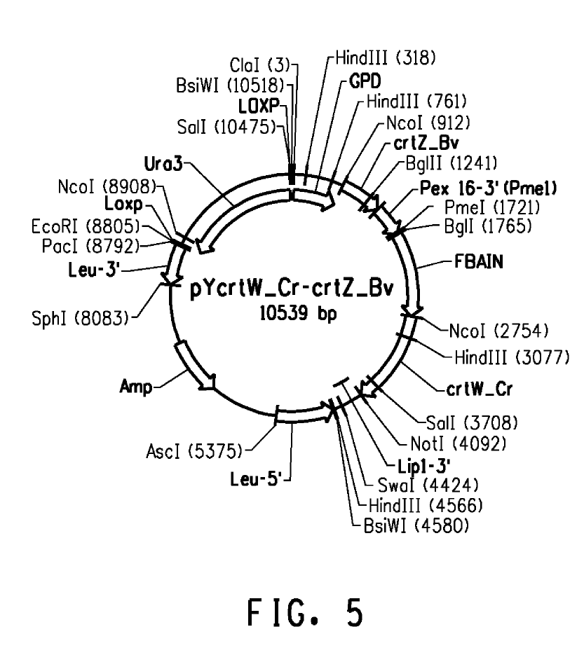

FIG. 5 is a plasmid map for pYcrtW_Cr-crtZ_Bv.

### BRIEF DESCRIPTION OF THE BIOLOGICAL SEQUENCES

The following sequences comply with 37 C.F.R. §1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822. The sequences are provided in the Table 1.

TABLE 1
Summary Of Nucleic Acid And Protein SEQ ID Numbers

| | Nucleic | Protein |
|---|---|---|
| | acid | SEQ ID |
| Description and Abbreviation | SEQ ID NO. | NO. |
| Plasmid pZKIeuN-6EP | 1 | — |
| Plasmid pYcrtEBI. | 2 | — |
| Plasmid pZKUGPE1S-P | 3 | — |
| Plasmid pYcrtEBIY | 4 | — |
| Coding sequence of geranylgeranyl | 5 | 6 |
| pyrophosphate synthase derived from | | |
| Enterobacteriaceae sp. DC413, codon-optimized | | |
| for expression in Yarrowia lipolytica (“crtE”) | | |
| FBAIN promoter for expression of crtE | 7 | — |
| LIP1-3′ terminator for expression of crtE | 8 | — |
| Coding sequence of phytoene synthase derived | 9 | 10 |
| from Enterobacteriaceae sp. DC413, codon- | | |
| optimized for expression in Yarrowia lipolytica | | |
| (“crtB”) | | |
| GDP PRO + Intron promoter for expression of | 11 | — |
| crtB | | |
| LIP2-3′ terminator for expression of crtB | 12 | — |
| Coding sequence of phytoene desaturase gene | 13 | 14 |
| derived from Enterobacteriaceae sp. DC413, | | |
| codon-optimized for expression in Yarrowia | | |
| lipolytica (“crtI”) | | |
| EXP promoter for expression of crtI | 15 | — |
| OCT terminator for expression of crtI | 16 | — |
| Coding sequence of lycopene cyclase gene | 17 | 18 |
| derived from Enterobacteriaceae sp. DC413, | | |
| codon-optimized for expression in Yarrowia | | |
| lipolytica (“crtY”). | | |
| GPAT promoter for expression of crtY | 19 | — |
| PEX16-3′ terminator for expression of crtY | 20 | — |
| Coding sequence of β-carotene ketolase | 21 | 22 |
| (“crtW_{Cr}”, also referred to as “bkt”) derived from | | |
| Chlamydomonas reinhardtii | | |
| FBAIN promoter for expression of crtW_{Cr} β- | 23 | — |
| carotene ketolase from Chlamydomonas | | |
| reinhardtii | | |
| lip1-3 terminator for expression of crtW_{cr} β- | 24 | — |
| carotene ketolase from Chlamydomonas | | |
| reinhardtii | | |
| Coding sequence for β-carotene hydroxylase | 25 | 26 |
| derived from Brevundimonas vesicularis, codon- | | |
| optimized for expression in Yarrowia lipolytica | | |
| (“crtZ_{Bv}”) | | |
| GPD promoter for expression of crtZ from | 27 | — |
| Brevundimonas vesicularis | | |
| pex16_3 terminator for expression of crtZ from | 28 | — |
| Brevundimonas vesicularis | | |
| Coding sequence for β-carotene hydroxylase | 29 | 30 |
| derived from Arabidopsis thaliana, codon- | | |
| optimized for expression in Yarrowia lipolytica | | |
| (“crtZ_{At}”) | | |
| GDP promoter for expression of crtZ_{At} | 31 | — |
| PEX16-3′ terminator for expression of crtZ_{At} | 32 | — |
| PCR primer SKS001 | 33 | — |
| PCR primer SKS002 | 34 | — |
| PCR primer SKS007 | 35 | — |
| PCR primer SKS008 | 36 | — |
| Plasmid pYcrtW_{Cr}-CrtZ_{Bv} | 37 | — |
| Plasmid pYcrtW_{Cr}-CrtZ_{At} | 38 | — |

### DETAILED DESCRIPTION OF THE INVENTION

In this disclosure, a number of terms and abbreviations are used.

The following definitions are provided.

The term “invention” or “present invention” as used herein is not meant to be limiting to any one specific embodiment of the invention but applies generally to any and all embodiments of the invention as described in the claims and specification.

As used herein, the articles “a”, “an”, and “the” preceding an element or component of the invention are intended to be nonrestrictive regarding the number of instances (i.e., occurrences) of the element or component. Therefore “a”, “an”, and “the” should be read to include one or at least one, and the singular word form of the element or component also includes the plural unless the number is obviously meant to be singular.

As used herein, the term “comprising” means the presence of the stated features, integers, steps, or components as referred to in the claims, but that it does not preclude the presence or addition of one or more other features, integers, steps, components or groups thereof. The term “comprising” is intended to include embodiments encompassed by the terms “consisting essentially of” and “consisting of”. Similarly, the term “consisting essentially of” is intended to include embodiments encompassed by the term “consisting of”.

As used herein, the term “about” modifying the quantity of an ingredient or reactant employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities. In one aspect, the term “about” means within 20% of the recited numerical value, preferably within 10%, and most preferably within 5%.

Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like.

As used herein, a “metabolic pathway” or “biosynthetic pathway”, in a biochemical sense, can be regarded as a series of chemical reactions occurring within a cell, catalyzed by enzymes, to achieve the formation of a defined product. Many of these pathways are elaborate, and involve a step by step modification of the initial substance to shape it into a product having the exact chemical structure desired. The present application describes carotenoid biosynthetic pathway. As used herein, the “R-carotene biosynthesis pathway” refers to the set of genes necessary to produce β-carotene from farnesyl pyrophosphate (farnesyl diphosphate; FPP). The genes necessary to produce β-carotene in the host cell can endogenous or foreign to the host cell so long as β-carotene produced. As used herein, the “set of β-carotene biosynthesis pathway genes” will refer to the combination of genes expressed within the host cell necessary to product β-carotene. In one embodiment, the set of β-carotene biosynthesis pathway genes is at least on expressible copy of the following: crtE (encoding “CrtE”; geranylgeranyl diphosphate synthase), crtB (encoding “CrtB”; phytoene synthase); crtI (encoding “CrtI”; phytoene desaturase); and crtY (encoding “CrtY”; lycopene cyclase) (See FIG. 1). In one embodiment, the β-carotene producing microbial host cell is a recombinant microbial host cell engineered to express the genes necessary to produce β-carotene from farnesyl diphosphate. In a further embodiment, the β-carotene-producing recombinant microbial host cell was engineered to express a combination of genes encoding geranylgeranyl diphosphate synthase, phytoene synthase, phytoene desaturase, and lycopene cyclase. The production of astaxanthin from β-carotene typically requires 2 additional enzymes, at least one 3-carotene ketolase (also referred to herein as a “carotenoid ketolase”) and at least one 3-carotene hydroxylase (also referred to herein as a “carotenoid hydroxylase”). In one embodiment, the “astaxanthin biosynthesis pathway” comprises the 3-carotene biosynthesis pathway genes plus (1) at least one gene encoding a carotenoid ketolase, and (2) at least one gene encoding a carotenoid hydroxylase (see FIG. 1).

The term “isoprenoid compound” refers to compounds formally derived from isoprene (2-methylbuta-1,3-diene; CH₂═C(CH₃)CH═CH₂), the skeleton of which can generally be discerned in repeated occurrence in the molecule. These compounds are produced biosynthetically via the isoprenoid pathway beginning with isopentenyl pyrophosphate (IPP) and formed by the head-to-tail condensation of isoprene units, leading to molecules which may be, for example, of 5, 10, 15, 20, 30, or 40 carbons in length.

As used herein, the term “carotenoid” refers to a class of hydrocarbons having a conjugated polyene carbon skeleton formally derived from isoprene. This class of molecules is composed of triterpenes (C₃₀ diapocarotenoids) and tetraterpenes (C₄₀ carotenoids) and their oxygenated derivatives; and, these molecules typically have strong light absorbing properties and may range in length in excess of C₂₀₀.

The term “carotenoid” may include both carotenes and xanthophylls. A “carotene” refers to a hydrocarbon carotenoid (e.g., phytoene, β-carotene and lycopene). In contrast, the term “xanthophyll” refers to a C₄₀ carotenoid that contains one or more oxygen atoms in the form of hydroxy-, methoxy-, oxo-, epoxy-, carboxy-, or aldehydic functional groups. Examples of xanthophylls include, but are not limited to antheraxanthin, adonixanthin, astaxanthin (i.e., 3,3″-dihydroxy-β,β-carotene-4,4″-dione), canthaxanthin (i.e., β,β-carotene-4,4″-dione), β-cryptoxanthin, keto-γ-carotene, echinenone, 3-hydroxyechinenone, 3′-hydroxyechinenone, zeaxanthin, adonirubin, tetrahydroxy-β,β′-caroten-4,4′-dione, tetrahydroxy-β,β′-caroten-4-one, caloxanthin, erythroxanthin, nostoxanthin, flexixanthin, 3-hydroxy-γ-carotene, 3-hydroxy-4-keto-γ-carotene, bacteriorubixanthin, bacteriorubixanthinal and lutein.

The term “functionalized” or “functionalization” refers to the (i) hydrogenation, (ii) dehydrogenation, (iii) cyclization, (iv) oxidation, or (v) esterification/glycosylation of any portion of the carotenoid backbone. This backbone is defined as the long central chain of conjugated double bonds. Functionalization may also occur by any combination of the above processes, to thereby result in creation of an acyclic carotenoid or a carotenoid terminated with one (monocyclic) or two (bicyclic) cyclic end groups. Additionally, some carotenoids arise from rearrangements of the carbon skeleton, or by the (formal) removal of part of the backbone structure.

All “tetraterpenes” or “C₄₀ carotenoids” consist of eight isoprenoid units joined in such a manner that the arrangement of isoprenoid units is reversed at the center of the molecule so that the two central methyl groups are in a 1,6-positional relationship and the remaining nonterminal methyl groups are in a 1,5-positional relationship. All C₄₀ carotenoids may be formally derived from the acyclic C₄₀H₅₆ structure, having a long central chain of conjugated double bonds that is subjected to various funcationalizations.

The term “CrtE” refers to a geranylgeranyl pyrophosphate synthase enzyme encoded by the crtE gene and which converts trans-trans-farnesyl diphosphate and IPP to pyrophosphate and geranylgeranyl diphosphate.

The term “CrtB” refers to a phytoene synthase enzyme encoded by the crtB gene which catalyzes the reaction from prephytoene diphosphate to phytoene.

The term “CrtI” refers to a phytoene desaturase enzyme encoded by the crtI gene. CrtI converts phytoene into lycopene via the intermediaries of phytofluene, ζ-carotene and neurosporene by the introduction of 4 double bonds.

The term “CrtY” refers to a lycopene cyclase enzyme encoded by the crtY gene that converts lycopene to 3-carotene.

The term “CrtZ” refers to a carotenoid hydroxylase enzyme (also referred to herein as a “β-carotene hydroxylase”) encoded by the crtZ gene that catalyzes a hydroxylation reaction. The oxidation reaction adds a hydroxyl group to cyclic carotenoids having a β-ionone type ring. It is known that CrtZ hydroxylases typically exhibit substrate flexibility, enabling production of a variety of hydroxylated carotenoids depending upon the available substrates; for example, CrtZ catalyzes the hydroxylation reaction from β-carotene to zeaxanthin.

The term “CrtW” refers to a β-carotene ketolase (also referred to herein as a “carotenoid ketolase” or “Bkt”) enzyme encoded by the crtW (bkt) gene that catalyzes an oxidation reaction where a keto group is introduced on the β-ionone type ring of cyclic carotenoids. This reaction converts cyclic carotenoids, such as β-carotene or zeaxanthin, into the ketocarotenoids canthaxanthin or astaxanthin, respectively. Intermediates in the process typically include echinenone and adonixanthin. It is known that CrtW ketolases typically exhibit substrate flexibility, enabling production of a variety of ketocarotenoids depending upon the available substrates.

The term “pigment” refers to a substance used for coloring another material. With respect to the present invention, the pigments described herein are carotenoids produced by a recombinant microbial host cell. These carotenoids can be used for coloring, for example, animal tissues (e.g., shrimp, salmonid fish, chicken skin, egg yolks).

The term “oleaginous” refers to those organisms that tend to store their energy source in the form of lipid (Weete, John D. In: Lipid Biochemistry of Fungi and other Organisms, Plenum, New York, N.Y., 1980). The term “oleaginous yeast” refers to those microorganisms classified as yeasts that can make oil. It is not uncommon for oleaginous microorganisms to accumulate in excess of about 25% of their dry cell weight as oil. Examples of oleaginous yeast include, but are no means limited to, the following genera: Yarrowia, Candida, Rhodotorula, Rhodosporidium, Cryptococcus, Trichosporon and Lipomyces. In one embodiment, the present recombinant microbial host cell is an oleaginous yeast. In a further embodiment, the present recombinant microbial host cell is a strain of Yarrowia lipolytica.

As used herein, an “isolated nucleic acid fragment” or “genetic construct” is a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA.

The term “complementary” is used to describe the relationship between nucleotide bases that are capable of hybridizing to one another. For example, with respect to DNA, adenosine is complementary to thymine and cytosine is complementary to guanine.

“Codon degeneracy” refers to the nature in the genetic code permitting variation of the nucleotide sequence without affecting the amino acid sequence of an encoded polypeptide. The skilled artisan is well aware of the “codon-bias” exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell.

“Chemically synthesized”, as related to a sequence of DNA, means that the component nucleotides were assembled in vitro. Manual chemical synthesis of DNA may be accomplished using well-established procedures or, automated chemical synthesis can be performed using one of a number of commercially available machines. “Synthetic genes” can be assembled from oligonucleotide building blocks that are chemically synthesized using procedures known to those skilled in the art. These building blocks are ligated and annealed to form gene segments that are then enzymatically assembled to construct the entire gene. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell, where sequence information is available. For example, the codon usage profile for Yarrowia lipolytica is provided in U.S. Pat. No. 7,125,672.

“Gene” refers to a nucleic acid fragment that expresses a specific protein, and that may refer to the coding region alone or may include regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene that is introduced into the host organism by gene transfer. Foreign genes can comprise native genes inserted into a non-native organism, native genes introduced into a new location within the native host, or chimeric genes. A “transgene” is a gene that has been introduced into the genome by a transformation procedure. A “codon-optimized gene” is a gene having its frequency of codon usage designed to mimic the frequency of preferred codon usage of the host cell.

“Coding sequence” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence (or located within an intron thereof), and which influence the transcription, RNA processing or stability, or translation of the associated coding sequence. Regulatory sequences may include promoters, translation leader sequences, introns, polyadenylation recognition sequences, RNA processing sites, effector binding sites and stem-loop structures.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters that cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The terms “3′ non-coding sequences” and “transcription terminator” refer to DNA sequences located downstream of a coding sequence. This includes polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The 3′ region can influence the transcription, RNA processing or stability, or translation of the associated coding sequence.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., the coding sequence is under the transcriptional control of the promoter). Coding sequences can be operably linked to regulatory sequences in sense or antisense orientation.

The term “expression”, as used herein, refers to the transcription and stable accumulation of sense (mRNA) or antisense RNA derived from the nucleic acid fragments of the invention. Expression may also refer to translation of mRNA into a polypeptide.

“Transformation” refers to the transfer of a nucleic acid molecule into a host organism, resulting in genetically stable inheritance. The nucleic acid molecule may be a plasmid that replicates autonomously, for example, or, it may integrate into the genome of the host organism. Host organisms containing the transformed nucleic acid fragments are referred to as “transgenic” or “recombinant” or “transformed” organisms.

The terms “plasmid” and “vector” refer to an extra chromosomal element often carrying genes that are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing an expression cassette(s) into a cell.

The term “expression cassette” refers to a fragment of DNA comprising the coding sequence of a selected gene and regulatory sequences preceding (5′ non-coding sequences) and following (3′ non-coding sequences) the coding sequence that are required for expression of the selected gene product. Thus, an expression cassette is typically composed of: (1) a promoter sequence; (2) a coding sequence; and, (3) a 3′ untranslated region (i.e., a terminator) that, in eukaryotes, usually contains a polyadenylation site. The expression cassette(s) is usually included within a vector, to facilitate cloning and transformation. Different expression cassettes can be transformed into different organisms including bacteria, yeast, plants and mammalian cells, as long as the correct regulatory sequences are used for each host.

As used herein, the term “expressible genetic construct” will refer a genetic fusion construct comprising a promoter operably linked to a coding sequence from a foreign gene and an appropriate terminator sequence. The promoter and terminator operably linked to the foreign coding sequence will likely be selected based on the type of recombinant host cell used. A recombinant host cell comprising an expressible genetic construct will be capable of expressing chimeric gene to produce the defined polypeptide or protein, such as an enzyme. As demonstrated in the working examples, several genes involved in the biosynthesis of astaxanthin were engineered into a recombinant microbial host cell. The coding sequences of these genes were operably linked to promoters and/or terminators suitable for expression in the microbial host cell. In one embodiment, the expressible genetic construct is described using the following format: promoter::coding sequence of the desired gene::terminator. For example, GPAT::crtY::PEX16-3′ refers to the expressible genetic construct comprising a GPAT promoter operably linked to the coding sequence from a foreign crtY gene which is operably linked to a PEX16-3′ terminator.

As used herein, the term “chromosomal integration” means that a chromosomal integration vector becomes congruent with the chromosome of a microorganism through recombination between homologous DNA regions on the chromosomal integration vector and within the chromosome.

As used herein, the term “chromosomal integration vector” means an extra-chromosomal vector that is capable of integrating into the host's genome through homologous recombination.

The term “sequence analysis software” refers to any computer algorithm or software program that is useful for the analysis of nucleotide or amino acid sequences. “Sequence analysis software” may be commercially available or independently developed. Typical sequence analysis software will include, but is not limited to: 1.) the GCG suite of programs (Wisconsin Package Version 9.0, Genetics Computer Group (GCG), Madison, Wis.); 2.) BLASTP, BLASTN, BLASTX (Altschul et al., J. Mol. Biol., 215:403-410 (1990)); 3.) DNASTAR (DNASTAR, Inc. Madison, Wis.); 4.) Sequencher (Gene Codes Corporation, Ann Arbor, Mich.); and, and 5.) the FASTA program incorporating the Smith-Waterman algorithm (W. R. Pearson, Comput. Methods Genome Res., [Proc. Int. Symp.] (1994), Meeting Date 1992, 111-20. Editor(s): Suhai, Sandor. Plenum: New York, N.Y.). Within the context of this application it will be understood that where sequence analysis software is used for analysis, that the results of the analysis will be based on the “default values” of the program referenced, unless otherwise specified. As used herein, “default values” will mean any set of values or parameters (as set by the software manufacturer) which originally load with the software when first initialized.

Standard recombinant DNA and molecular cloning techniques used herein are well known in the art and are described by Sambrook, J., Fritsch, E. F. and Maniatis, T., Molecular Cloning: A Laboratory Manual, 3^{rd} ed., Cold Spring Harbor Laboratory: Cold Spring Harbor, N.Y. (2001) (hereinafter “Maniatis”); by Silhavy, T. J., Bennan, M. L. and Enquist, L. W., Experiments with Gene Fusions, Cold Spring Harbor Laboratory: Cold Spring Harbor, N.Y. (1984); and by Ausubel, F. M. et al., Current Protocols in Molecular Biology, published by Greene Publishing Assoc. and Wiley-Interscience, Hoboken, N.J. (1987).

### Microbial Hosts for Carotenoid Production

The genes and gene products of the instant sequences may be produced in heterologous host cells, particularly in microbial host cells. Preferred microbial host cells for expression of the chimeric genes are microbial hosts that can be found within the fungal or bacterial families and which grow over a wide range of temperature, pH values, and solvent tolerances. For example, it is contemplated that any of bacteria, yeast, and filamentous fungi may suitably host the expression of the present nucleic acid molecules. Examples of host strains include, but are not limited to, bacterial, fungal or yeast species such as Aspergillus, Trichoderma, Saccharomyces, Pichia, Phaffia, Kluyveromyces, Candida, Hansenula, Yarrowia, Salmonella, Bacillus, Acinetobacter, Zymomonas, Agrobacterium, Erythrobacter, Chlorobium, Chromatium, Flavobacterium, Cytophaga, Rhodobacter, Rhodococcus, Streptomyces, Brevibacterium, Corynebacteria, Mycobacterium, Deinococcus, Escherichia, Erwinia, Pantoea, Pseudomonas, Sphingomonas, Methylomonas, Methylobacter, Methylococcus, Methylosinus, Methylomicrobium, Methylocystis, Alcaligenes, Synechocystis, Synechococcus, Anabaena, Thiobacillus, Methanobacterium, Klebsiella, and Myxococcus. In one embodiment, bacterial host strains include Escherichia, Bacillus, Kluyveromyces, and Pseudomonas. In another embodiment, the recombinant microbial host cell is a recombinant fungal cell. In a further embodiment, the fungal cell is a yeast is selected form the genera Phaffia/Xanthophyllomyces, Saccharomyces, Thraustochytrium, Yarrowia, Candida, Rhodotorula, Rhodosporidium, Cryptococcus, Trichosporon, Lipomyces and Labyrinthula. In a preferred aspect, the recombinant microbial host cell is a member of the genera Yarrowia; preferably a strain of Yarrowia lipolytica.

In one embodiment, the yeast may be oleaginous yeast. Oleaginous organisms are those organisms that tend to store their energy source in the form of lipid (Weete, John D., supra). Generally, the cellular oil content of these microorganisms follows a sigmoid curve, wherein the concentration of lipid increases until it reaches a maximum at the late logarithmic or early stationary growth phase and then gradually decreases during the late stationary and death phases (Yongmanitchai and Ward, Appl. Environ. Microbiol., 57:419-25 (1991)).

The term “oleaginous yeast” refers to those microorganisms classified as yeasts that can accumulate in excess of about 25% of their dry cell weight (dcw) as oil, more preferably greater than about 30% of the dcw, and most preferably greater than about 40% of the dcw under oleaginous conditions. In one embodiment, the present recombinant microbial host cell is oleaginous yeast selected from Yarrowia, Candida, Rhodotorula, Rhodosporidium, Cryptococcus, Trichosporon and Lipomyces. In a further embodiment, the oleaginous yeast is Rhodosporidium toruloides, Liopmyces starkeyii, L. lipoferus, Candida revkaufi, C. pulcherrima, C. tropicalis, C. utilis, Trichosporon pullans, T. cutaneum, Rhodotorula glutinus, R. graminis or Yarrowia lipolytica (formerly classified as Candida lipolytica). The technology for growing oleaginous yeast with high oil content is well developed (for example, see EP0005277B1; Ratledge, C., Prog. Ind. Microbiol., 16:119-206 (1982)); and, these organisms have been commercially used for a variety of purposes in the past.

### Carotenoid Production

The genetics of carotenoid biosynthesis are well known (Armstrong, G., in Comprehensive Natural Products Chemistry Volume 2: Isoprenoids Including Carotenoids and Steroids., Elsevier, pp 321-352 (1999), Oxford, UK); Lee, P. and Schmidt-Dannert, C., Appl. Microbiol. Biotechnol., 60:1-11 (2002); Lee et al., Chem. Biol., 10:453-462 (2003); Fraser, P. and Bramley, P., Progress in Lipid Research, 43:228-265 (2004)). This pathway is extremely well studied in the Gram-negative, pigmented bacteria of the genera Pantoea, formerly known as Erwinia. Of particular interest are the genes responsible for the production of C₄₀ carotenoids used as pigments in animal feeds (e.g., zeaxanthin, lutein, canthaxanthin and astaxanthin).

The enzymatic pathway involved in the biosynthesis of carotenoid compounds can be conveniently viewed in two parts: the upper isoprenoid pathway (isoprenoid biosynthesis is found in all organisms) providing farnesyl pyrophosphate (FPP); and, the lower carotenoid biosynthetic pathway (found in a subset of organisms), which converts FPP to C₄₀ carotenoids.

### Farnesyl Pyrophosphate Synthesis Via the Mevalonate Pathway:

The upper isoprenoid biosynthetic pathway leads to the production of the C₅ isoprene subunit, isopentenyl pyrophosphate (IPP). This biosynthetic process may occur through the mevalonate pathway (from acetyl CoA) or the non-mevalonate pathway (from pyruvate and glyceraldehyde-3-phosphate). The non-mevalonate pathway has been characterized in bacteria, green algae and higher plants, but not in yeast and animals (Horbach et al., FEMS Microbiol. Lett., 111:135-140 (1993); Rohmer et al., Biochem., 295:517-524 (1993); Schwender et al., Biochem., 316:73-80 (1996); and, Eisenreich et al., Proc. Natl. Acad. Sci. U.S.A., 93:6431-6436 (1996)).

Yeasts and animals typically use the mevalonate pathway to produce IPP, which is subsequently converted to farnesyl diphosphate; FPP (C₁₅). In this pathway, 2 molecules of acetyl-CoA are condensed by thiolase to yield acetoacetyl-CoA, which is subsequently converted to 3-hydroxy-3-methylglutaryl-CoA (HMG-CoA) by the action of 3-hydroxymethyl-3-glutaryl-CoA synthase (HMG-CoA synthase). Next, 3-hydroxy-3-methylglutaryl-CoA reductase (HMG-CoA reductase; the rate controlling step in the mevalonate pathway) converts HMG-CoA to mevalonate, to which 2 molecules of phosphate residues are then added by the action of 2 kinases (i.e., mevalonate kinase and phosphomevalonate kinase, respectively). Mevalonate pyrophosphate is then decarboxylated by the action of mevalonate pyrophosphate decarboxylase to yield IPP, which becomes the building unit for a wide variety of isoprene molecules necessary in living organisms.

IPP is isomerized to dimethylaryl pyrophosphate (DMAPP) by the action of IPP isomerase. IPP and DMAPP are then converted to the C₁₀ unit geranyl pyrophosphate (GPP) by a head to tail condensation. In a similar condensation reaction between GPP and IPP, GPP is converted to the C₁₅ unit FPP, an important substrate in ergosterol biosynthesis in yeast. The biosynthesis of GPP and FPP from IPP and DMAPP is catalyzed by the enzyme FPP synthase.

Carotenoid Biosynthesis from Farnesyl Pyrophosphate:

Although the enzymatic pathway involved in the biosynthesis of carotenoid compounds converts FPP to a suite of carotenoids, the C₄₀ pathway can be subdivided into two parts comprising: (1) the C₄₀ backbone genes (i.e., crtE, crtB, crtI, and crtY) encoding enzymes responsible for converting FPP to β-carotene; and, (2) subsequent functionalization genes (e.g., crtW/bkt/crtO, crtR, crtX and crtZ, responsible for adding various functional groups to the β-ionone rings of β-carotene; and, Lut1, responsible for adding a hydroxyl group to α-carotene) (FIG. 1).

More specifically, the carotenoid biosynthetic pathway begins with the conversion of FPP to geranylgeranyl pyrophosphate (GGPP). In this first step, the enzyme geranylgeranyl pyrophosphate synthase (encoded by the crtE gene) condenses the C₁₅ FPP with IPP, creating the C₂₀ compound GGPP. Next, a phytoene synthase (encoded by the gene crtB) condenses two GGPP molecules to form phytoene, the first C₄₀ carotenoid compound in the pathway. Subsequently, a series of sequential desaturations (i.e., producing the intermediaries of phytofluene, ζ-carotene and neurosporene) occur, catalyzed by the enzyme phytoene desaturase (encoded by the gene crtI) and resulting in production of lycopene. Finally, the enzyme lycopene cyclase (encoded by the gene crtY) forms β-ionone rings on each end of lycopene, forming the bicyclic carotenoid β-carotene.

The rings of β-carotene can subsequently be functionalized by a carotenoid ketolase (encoded by the genes crtW, crtO or bkt) and/or carotenoid hydroxylase (encoded by the genes crtZ or crtR) forming commercially important xanthophyll pigments such as canthaxanthin, astaxanthin and zeaxanthin. The pathway from β-carotene to astaxanthin is somewhat non-linear in nature as a variety of intermediates can be formed (FIG. 1).

As used herein, the phrases “without a measurable concomitant accumulation of ketolated or hydroxylated intermediates” and “does not concomitantly accumulate a significant amount of adonixanthin, zeaxanthin, adonirubin, echinenone, 3-hydroxyechinenone, 3′-hydroxyechinenone, canthaxanthin or β-cryptoxanthin” will refer to a recombinant host cell expressing the present specified combination of β-carotene ketolases and β-carotene hydroxylases that facilitates production of astaxanthin without a significant concomitant accumulation of ketolated and hydroxylated intermediates. As used herein, “ketolated and hydroxylated intermediates” refers to any one of adonixanthin, zeaxanthin, adonirubin, echinenone, 3-hydroxyechinenone, 3′-hydroxyechinenone, canthaxanthin or β-cryptoxanthin. As such, these ketolated and/or hydroxylated carotenoids are “intermediates” in the pathway between β-carotene and astaxanthin.

In one embodiment, the phrase “significant amount of a ketolated or hydroxylated intermediate” will be defined as a ketolated or hydroxylated intermediate to astaxanthin ratio (measured as ppm (dcw)) of 0.015 or more, preferably 0.013 or more, more preferably 0.01 or more, and most preferably 0.007 or more. As demonstrated in the present examples (see Tables 11 and 12), a concentration of astaxanthin exceeding 150 ppm (dcw) was obtainable in multiple strains without a detectable amount (limit of detection of less than 2 ppm (dcw)) of any one of the following ketolated and/or hydroxylated intermediates: adonixanthin, zeaxanthin, adonirubin, echinenone, 3-hydroxyechinenone, 3′-hydroxyechinenone, canthaxanthin, and β-cryptoxanthin. As such, approximately 2 ppm (ketolated or hydroxylated intermediate)/150 ppm astaxanthin is approximately 0.013. Several strains produced astaxanthin concentrations as high as 276 ppm dry cell weight (AX165) and 297 ppm dcw (AX265) without a detectable concentration (limit of detection of less than 2 ppm) of any one of the ketolated and/or hydroxylated intermediates. As such, a ratio of 2 ppm/276 ppm astaxanthin or 2 ppm/297 ppm astaxanthin were calculated to be approximately 0.007. Conversely, the ratio of astaxanthin to ketolated and/or hydroxylated intermediate (referred to herein as the “astaxanthin:hydroxylated and/or ketolated intermediate ratio” or simply the “astaxanthin:intermediate ratio”) is measured as ppm dry cell weight and is at least 75:1, preferably at least 100:1, more preferably at least 125:1 and most preferably 150:1. In another embodiment, the phrase “without a significant amount of a ketolated or hydroxylated intermediate” will refer to a recombinant microbial host cell expressing the present combination of β-carotene ketolase and β-carotene hydroxylase which is capable of producing at least 150 ppm astaxanthin, preferably at least 200 ppm, more preferably at least 250 ppm, and most preferably at least 275 ppm astaxanthin (dcw) without concomitantly accumulating 2 ppm or more (dcw) of any one of the following ketolated and/or hydroxylated carotenoid intermediates: adonixanthin, zeaxanthin, adonirubin, echinenone, 3-hydroxyechinenone, 3′-hydroxyechinenone, canthaxanthin or β-cryptoxanthin.

### Microbial Expression Systems, Cassettes & Vectors, and Transformation

Microbial expression systems and expression vectors containing regulatory sequences that direct high level expression of foreign proteins are well known to those skilled in the art. Any of these could be used to construct chimeric genes for production of the desired compound(s) (i.e., carotenoids). These chimeric genes could then be introduced into appropriate microorganisms via transformation to allow for high level expression of the enzymes.

Vectors (e.g., constructs, plasmids) and DNA expression cassettes useful for the transformation of suitable host cells are well known in the art. The specific choice of sequences present in the construct is dependent upon the desired expression products, the nature of the host cell, and the proposed means of separating transformed cells versus non-transformed cells. Typically, however, the vector contains at least one expression cassette, a selectable marker and sequences allowing autonomous replication or chromosomal integration. Suitable expression cassettes comprise a region 5′ of the gene that controls transcriptional initiation (e.g., a promoter), the gene coding sequence, and a region 3′ of the DNA fragment that controls transcriptional termination (i.e., a terminator). It is most preferred when both control regions are derived from genes from the transformed host cell, although it is to be understood that such control regions need not be derived from the genes native to the specific species chosen as a production host.

Initiation control regions or promoters, which are useful to drive expression of the relevant genes in the desired yeast host cell are numerous and familiar to those skilled in the art. Virtually any promoter capable of directing expression of these genes in the selected host cell is suitable for the present invention. Expression in a host cell can be accomplished in a transient or stable fashion. Transient expression can be accomplished by inducing the activity of a regulatable promoter operably linked to the gene of interest. Stable expression can be achieved by the use of a constitutive promoter operably linked to the gene of interest. As an example, when the host cell is yeast, transcriptional and translational regions functional in yeast cells are provided, particularly from the host species (e.g., see U.S. Pat. No. 7,238,482 and U.S. Patent Appl. Pub. No. 2006-0115881A1] for preferred transcriptional initiation regulatory regions for use in Yarrowia lipolytica). Any one of a number of regulatory sequences can be used, depending upon whether constitutive or induced transcription is desired, the efficiency of the promoter in expressing the ORF of interest, the ease of construction and the like.

Nucleotide sequences surrounding the translational initiation codon ‘ATG’ have been found to affect expression in yeast cells. If the desired polypeptide is poorly expressed in yeast, the nucleotide sequences of exogenous genes can be modified to include an efficient yeast translation initiation sequence to obtain optimal gene expression. For expression in yeast, this can be done by site-directed mutagenesis of an inefficiently expressed gene by fusing it in-frame to an endogenous yeast gene, preferably a highly expressed gene. Alternatively, as demonstrated in Yarrowia lipolytica, one can determine the consensus translation initiation sequence in the host and engineer this sequence into heterologous genes for their optimal expression in the host of interest (U.S. Pat. No. 7,125,672).

Termination control regions may be derived from various genes native to the preferred hosts. Optionally, a termination site may be unnecessary; however, it is most preferred if included. As used herein, the termination region can be derived from the 3′ region of the gene from which the initiation region was obtained or from a different gene. A large number of termination regions are known and function satisfactorily in a variety of hosts (when utilized both in the same and different genera and species from where they were derived). Typically, the termination region usually is selected more as a matter of convenience rather than because of any particular property. For the purposes herein, when the host cell is a yeast the termination region is preferably derived from a yeast gene, particularly Saccharomyces, Schizosaccharomyces, Candida, Yarrowia or Kluyveromyces. The 3′-regions of mammalian genes encoding γ-interferon and α-2 interferon are also known to function in yeast. Although not intended to be limiting, preferred termination regions useful in the disclosure herein include: ˜100 bp of the 3′ region of the Yarrowia lipolytica extracellular protease (Xpr; GENBANK® Accession No. M17741); the acyl-CoA oxidase (Aco3: GENBANK® Accession No. AJ001301 and No. CAA04661; Pox3: GENBANK® Accession No. XP_{—}503244) terminators; the Pex20 (GENBANK® Accession No. AF054613) terminator; the Pex16 (GENBANK® Accession No. U75433) terminator; the Lip1 (GENBANK® Accession No. Z50020) terminator; the Lip2 (GENBANK® Accession No. AJ012632) terminator; and the 3-oxoacyl-coA thiolase (Oct; GENBANK® Accession No. X69988) terminator.

Merely inserting a gene into a cloning vector does not ensure that it will be successfully expressed at the level needed. In response to the need for a high expression rate, many specialized expression vectors have been created by manipulating a number of different genetic elements that control aspects of transcription, translation, protein stability, oxygen limitation and secretion from the microbial host cell. More specifically, some of the molecular features that have been manipulated to control gene expression include: 1.) the nature of the relevant transcriptional promoter and terminator sequences; 2.) the number of copies of the cloned gene and whether the gene is plasmid-borne or integrated into the genome of the host cell; 3.) the final cellular location of the synthesized foreign protein; 4.) the efficiency of translation and correct folding of the protein in the host organism; 5.) the intrinsic stability of the mRNA and protein of the cloned gene within the host cell; and, 6.) the codon usage within the cloned gene, such that its frequency approaches the frequency of preferred codon usage of the host cell. Each type of these modifications is encompassed in the present invention as means to further optimize expression of the crt genes required herein. Methods of codon-optimizing foreign genes for optimal expression in Yarrowia lipolytica are set forth in U.S. Pat. No. 7,125,672.

Once the DNA encoding a polypeptide suitable for expression in an appropriate microbial host cell has been obtained, it is placed in a plasmid vector capable of autonomous replication in a host cell, or it is directly integrated into the genome of the host cell. Integration of expression cassettes can occur randomly within the host genome or can be targeted through the use of constructs containing regions of homology with the host genome sufficient to target recombination within the host locus. Where constructs are targeted to an endogenous locus, all or some of the transcriptional and translational regulatory regions can be provided by the endogenous locus.

Constructs comprising a coding region of interest may be introduced into a host cell by any standard technique. These techniques include transformation (e.g., lithium acetate transformation [Guthrie, C., Methods in Enzymology, 194:186-187 (1991)]), protoplast fusion, biolistic impact, electroporation, microinjection, or any other method that introduces the gene of interest into the host cell. More specific teachings applicable for yeast (i.e., Yarrowia lipolytica) include U.S. Pat. Nos. 4,880,741 and 5,071,764 and Chen, D. C. et al. (Appl. Microbiol. Biotechnol., 48(2):232-235 (1997)).

Where two or more genes are expressed from separate replicating vectors, it is desirable that each vector has a different means of selection and should lack homology to the other construct(s) to maintain stable expression and prevent reassortment of elements among constructs. Judicious choice of regulatory regions, selection means and method of propagation of the introduced construct(s) can be experimentally determined so that all introduced genes are expressed at the necessary levels to provide for synthesis of the desired products.

For convenience, a host cell that has been manipulated by any method to take up a DNA sequence (e.g., an expression cassette) will be referred to as “transformed” or “recombinant” herein. The transformed host will have at least one copy of the expression construct and may have two or more, depending upon whether the gene is integrated into the genome, amplified, or is present on an extrachromosomal element having multiple copy numbers.

The transformed host cell can be identified by various selection techniques, as described in U.S. Pat. No. 7,238,482 and U.S. Patent Appl. Pub. No. 2006-0115881A1. Preferred selection methods for use herein are resistance to kanamycin, hygromycin and the amino glycoside G418, as well as ability to grow on media lacking uracil, leucine, lysine, tryptophan or histidine. In alternate embodiments, 5-fluoroorotic acid (5-fluorouracil-6-carboxylic acid monohydrate; “5-FOA”) is used for selection of yeast Ura⁻ mutants. The compound is toxic to yeast cells that possess a functioning URA3 gene encoding orotidine 5′-monophosphate decarboxylase (OMP decarboxylase); thus, based on this toxicity, 5-FOA is especially useful for the selection and identification of Ura⁻ mutant yeast strains (Bartel, P. L. and Fields, S., Yeast 2-Hybrid System, (1997) Oxford University: New York, N.Y., vol. 7, pp. 109-147). More specifically, one can first knockout the native Ura3 gene to produce a strain having a Ura− phenotype, wherein selection occurs based on 5-FOA resistance. Then, a cluster of multiple chimeric genes and a new Ura3 gene can be integrated into a different locus of the Yarrowia genome to thereby produce a new strain having a Ura+ phenotype. Subsequent integration produces a new Ura3− strain (again identified using 5-FOA selection), when the introduced Ura3 gene is knocked out. Thus, the Ura3 gene (in combination with 5-FOA selection) can be used as a selection marker in multiple rounds of transformation.

### Microbial Fermentation Processes

The transformed microbial host cell is grown under conditions that optimize expression of chimeric genes and produce the greatest and the most economical yield of desired carotenoids. In general, media conditions that may be optimized include the type and amount of carbon source, the type and amount of nitrogen source, the carbon-to-nitrogen ratio, the oxygen level, growth temperature, pH, length of the biomass production phase, length of the oil accumulation phase and the time and method of cell harvest. Microorganisms of interest, such as yeast (e.g., Yarrowia lipolytica) are generally grown in complex media (e.g., yeast extract-peptone-dextrose broth (YPD)) or a defined minimal media that lacks a component necessary for growth and thereby forces selection of the desired expression cassettes (e.g., Yeast Nitrogen Base (DIFCO Laboratories, Detroit, Mich.)).

Fermentation media in the present invention must contain a suitable carbon source. Suitable carbon sources are taught in U.S. Pat. No. 7,238,482. Although it is contemplated that the source of carbon utilized in the present invention may encompass a wide variety of carbon-containing sources, preferred carbon sources are sugars, glycerol and/or fatty acids. Most preferred is glucose and/or fatty acids containing between 10-22 carbons.

Nitrogen may be supplied from an inorganic (e.g., (NH₄)₂SO₄) or organic (e.g., urea or glutamate) source. In addition to appropriate carbon and nitrogen sources, the fermentation media must also contain suitable minerals, salts, cofactors, buffers, vitamins and other components known to those skilled in the art suitable for the growth of the host and promotion of the enzymatic pathways necessary for carotenoid production.

Preferred growth media in the present invention are common commercially prepared media, such as Yeast Nitrogen Base (DIFCO Laboratories, Detroit, Mich.). Other defined or synthetic growth media may also be used and the appropriate medium for growth of the transformed host cells will be known by one skilled in the art of microbiology or fermentation science. A suitable pH range for the fermentation is typically between about pH 4.0 to pH 8.0, wherein pH 5.5 to pH 7.5 is preferred as the range for the initial growth conditions. The fermentation may be conducted under aerobic or anaerobic conditions, wherein microaerobic conditions are preferred.

### Purification and Processing of Carotenoids

In one embodiment, the primary product is yeast biomass. As such, isolation and purification of the carotenoid-containing oils from the biomass may not be necessary (i.e., wherein the biomass is the product).

However, certain end uses and/or product forms may require partial and/or complete isolation/purification of the carotenoid-containing oil from the biomass, to result in partially purified biomass, purified oil, and/or purified carotenoids. Given the lipophilic/hydrophobic nature of carotenoids, many techniques applied to isolate/purify microbially-produced oils should work to isolate carotenoids as well, especially when the desired product is a pigmented oil. As such, any number of well known techniques can be used to isolate the compounds from the biomass including, but not limited to: extraction (e.g., U.S. Pat. No. 6,797,303 and No. 5,648,564) with organic solvents, sonication, supercritical fluid extraction (e.g., using carbon dioxide), saponification and physical means such as presses, or combinations thereof. One is referred to the teachings of U.S. Pat. No. 7,238,482 for additional details.

Finally, one skilled in the art will be aware of the appropriate means to selectively purify a specific carotenoid from a carotenoid-containing mixture comprising various carotenoid intermediates in addition to the desired carotenoid.

### Use of Compositions Comprising Carotenoids

The carotenoids produced by the present processes may be used as pigments, antioxidants, or as both in various commercial product.

In some embodiments, the present invention is drawn to “pigmented microbial biomass/oils”, wherein the term pigmented microbial biomass/oils refers to a microbial biomass/oil of the invention comprising at least one carotenoid, wherein the carotenoid is present in an “effective” amount such that the final product and/or product formulation within which the pigmented microbial biomass/oil is incorporated becomes effectively pigmented. One of skill in the art of processing and formulation will understand how the amount and composition of the pigmented microbial biomass/oils may be added to the product and/or product formulation and how the “effective” amount will depend according to target species and/or end use (e.g., the food or feed product, cosmetic or personal care product, supplement, etc.). For example, an “effective amount of pigment” with respect to an animal feed refers to an amount that effectively pigments at least one animal tissue (e.g., chicken products such as egg yolks; crustacean muscle tissue and/or shell tissue; fish muscle tissue and/or skin tissue, etc.) under feeding conditions considered suitable for growth of the target animal species. The amount of pigment incorporated into the animal feed may vary according to target species. Typically, the amount of pigment product incorporated into the feed product takes into account pigmentation losses associated with feed processing conditions, typical handling and storage conditions, the stability of the pigment in the feed, the bioavailability/bioabsorption efficiency of the particular species, the pigmentation rate of the animal tissue targeted for pigmentation, and the overall profile of pigment isomers (wherein some are preferentially absorbed over others), to name a few.

In some embodiments, the invention provides an animal feed, food product, dietary supplement, pharmaceutical composition, infant formula, or personal care product comprising yeast biomass/oil comprising at least one carotenoid. In other words, the carotenoid product of the present invention is used as an ingredient in the final formulation of an animal feed, food product, dietary supplement, pharmaceutical composition, infant formula, or personal care product. It is contemplated that the pigmented and/or stabilized microbial biomass/oils of the invention comprising carotenoids will function in each of these applications to impart the health benefits of current formulations using more traditional sources of carotenoids. In some embodiments, yeast biomass comprises at least about 25 wt % oil, preferably at least about 30-40 wt %, and most preferably at least about 40-50 wt % microbially-produced oil.

### Food Products

Pigmented microbial biomass/oils of the invention comprising at least one carotenoid will be suitable for use in a variety of food and feed products including, but not limited to food analogs, meat products, cereal products, baked foods, snack foods and dairy products. Alternatively, the pigmented biomass/oils (or derivatives thereof) may be incorporated into cooking oils, fats or margarines formulated so that in normal use the recipient would receive the desired amount for dietary supplementation. The pigmented biomass/oils may also be incorporated into infant formulas, nutritional supplements or other food products and may find use as anti-inflammatory or cholesterol lowering agents.

The term “food product” refers to any food generally suitable for human consumption. Typical food products include but are not limited to meat products, cereal products, baked foods, snack foods, dairy products and the like. Meat products encompass a broad variety of products. In the United States “meat” includes “red meats” produced from cattle, hogs and sheep. In addition to the red meats there are poultry items which include chickens, turkeys, geese, guineas and ducks and the fish and shellfish. There is a wide assortment of seasoned and processed meat products: fresh, cured and fried, and cured and cooked. Sausages and hot dogs are examples of processed meat products. Thus, the term “meat products” as used herein includes, but is not limited to, processed meat products.

A cereal food product is a food product derived from the processing of a cereal grain. A cereal grain includes any plant from the grass family that yields an edible grain (seed). The most popular grains are barley, corn, millet, oats, quinoa, rice, rye, sorghum, triticale, wheat and wild rice. Examples of a cereal food product include, but are not limited to: whole grain, crushed grain, grits, flour, bran, germ, breakfast cereals, extruded foods, pastas and the like.

A baked goods product comprises any of the cereal food products mentioned above and has been baked or processed in a manner comparable to baking, i.e., to dry or harden by subjecting to heat. Examples of a baked good product include, but are not limited to: bread, cakes, doughnuts, bars, pastas, bread crumbs, baked snacks, mini-biscuits, mini-crackers, mini-cookies and mini-pretzels. As was mentioned above, pigmented microbial biomass/oils of the invention can be used as an ingredient.

### Animal Feed Products

Animal feeds are generically defined herein as products intended for use as feed or for mixing in feed for animals other than humans. More specifically, the term “animal feed” refers to feeds intended exclusively for consumption by animals, including domestic animals (e.g., pets, farm animals, home aquarium fish, etc.) or for animals raised for the production of food (e.g., poultry, eggs, fish, crustacea, etc.).

More specifically, although not limited therein, it is expected that the pigments and/or pigmented microbial biomass/oils can be used within pet food products, ruminant and poultry food products and aquaculture food products. Aquaculture food products (or “aquafeeds”) are those products intended to be used in aquafarming, which concerns the propagation, cultivation or farming of aquatic organisms and/or animals in fresh or marine waters. More specifically, the term “aquaculture” refers to the production and sale of farm raised aquatic plants and animals. Typical examples of animals produced through aquaculture include, but are not limited to: lobsters, shrimp, prawns, and fish (i.e., ornamental and/or food fish).

The pigments and/or pigmented microbial biomass/oils can be used as an ingredient in any of the animal feeds described above. In addition to providing necessary carotenoid pigments, the recombinant host cell itself is a useful source of protein and other nutrients (e.g., vitamins, minerals, nucleic acids, complex carbohydrates, etc.) that can contribute to overall animal health and nutrition, as well as increase a formulation's palatability.

In one embodiment, the pigmented animal feed is an animal feed selected from the group consisting of: fish feed, crustacea feed, shrimp feed, crab feed, lobster feed, and chicken feed. The nutritional requirements and feed forms for each animal feed are well known in the art (for example, see Nutrient Requirements of Fish, published by the Board of Agriculture's Committee on Animal Nutrition, National Research Council, National Academy: Washington, D.C. 1993; and Nutrient Requirements of Poultry, published by the Board of Agriculture's Committee on Animal Nutrition, National Research Council, National Academy: Washington, D.C. 1994).

Various means are available to incorporate the pigment and/or pigmented microbial biomass/oils into animal feed (typically in the form of feed pellets). For example, the biomass/oils can be incorporated into the feed mash prior to extrusion or after the extrusion process (“post-extrusion applied”) by mixing and dispersing the biomass/oils in a suitable oil that is subsequently applied to the pellet. Typically a “suitable oil” is fish oil (e.g., Capelin oil) or a vegetable oil (e.g., corn oil, sunflower oil, soybean oil, etc.), although in preferred embodiments the “suitable oil” is microbially produced.

Although the amount of total carotenoid incorporated into the post-extrusion prepared pigmented animal feed may be less than that found in pre-extrusion supplemented feed, the resulting preferential isomer content may be higher (e.g., the heat of the extrusion process may isomerize some pigments). It should be noted that many extrusion processes run at elevated temperatures sufficient to possibly degrade and/or alter carotenoids supplemented to the feed mash prior to extrusion. It is possible to use a cold extrusion process to circumvent this problem; however, the physical stability of the cold-extruded pellets tends to be inferior in comparison to the “hot-extruded” feed pellets.

The size and shape of the feed pellets may vary according to the target species and developmental stage. The amount of pigmented biomass product formulated into feed pellets can be adjusted and/or optimized for the particular application. Factors to consider include, but are not limited to: the concentration of the pigment in the biomass, the concentration of the pigment in the pigmentation product, the target species, the age and/or growth rate of the selected species, the type of carotenoid used, the bioabsorption characteristics of the chosen pigment in the context of the species to be pigmented, the feeding schedule, the cost of the pigment, and the palatability of the resulting feed. One of skill in the art can adjust the amount of pigment and/or pigmented microbial biomass/oil incorporated into the feed so that adequate levels of carotenoid are present while balancing the nutritional requirements of the species. Typical concentrations of the carotenoid pigment incorporated into, for example, fish feed range from about 10 to about 200 mg/kg of fish feed, wherein a preferred range is from about 10 mg/kg to about 100 mg/kg, a more preferred range is from about 10 mg/kg to about 80 mg/kg and a most preferred range is from about 20 mg/kg to about 60 mg/kg, depending on the specific product.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

All reagents and materials were obtained from DIFCO Laboratories (Detroit, Mich.), GIBCO/BRL (Gaithersburg, Md.), TCI America (Portland, Oreg.), Roche Diagnostics Corporation (Indianapolis, Ind.), Thermo Scientific (Pierce Protein Research Products) (Rockford, Ill.) or Sigma/Aldrich Chemical Company (St. Louis, Mo.), unless otherwise specified.

The following abbreviations in the specification correspond to units of measure, techniques, properties, or compounds as follows: “sec” or “s” means second(s), “min” means minute(s), “h” or “hr” means hour(s), “4” means microliter(s), “mL” means milliliter(s), “L” means liter(s), “mM” means millimolar, “M” means molar, “mmol” means millimole(s), “ppm” means part(s) per million, “wt” means weight, “wt %” means weight percent, “g” means gram(s), “mg” means milligram(s), “μg” means microgram(s), “ng” means nanogram(s), “g” means gravity, “HPLC” means high performance liquid chromatography, “dd H₂O” means distilled and deionized water, “dcw” means dry cell weight, “ATCC” or “ATCC®” means the American Type Culture Collection (Manassas, Va.), “U” means unit(s) of perhydrolase activity, “rpm” means revolution(s) per minute, “Tg” means glass transition temperature, and “EDTA” means ethylenediaminetetraacetic acid.

The structure of an expression cassette will be represented by a simple notation system of “X::Y::Z”, wherein X describes the promoter fragment, Y describes the gene fragment, and Z describes the terminator fragment, which are all operably linked to one another.

### General Methods

Standard recombinant DNA and molecular cloning techniques used herein are well known in the art and are described by Sambrook, J. and Russell, D., Molecular Cloning: A Laboratory Manual, Third Edition, Cold Spring Harbor Laboratory Press, Cold Spring Harbor, N.Y. (2001); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et. al., Short Protocols in Molecular Biology, 5^{th} Ed. Current Protocols and John Wiley and Sons, Inc., N.Y., 2002.

Materials and Methods suitable for the maintenance and growth of bacterial cultures are also well known in the art. Techniques suitable for use in the following Examples may be found in Manual of Methods for General Bacteriology, Phillipp Gerhardt, R. G. E. Murray, Ralph N. Costilow, Eugene W. Nester, Willis A. Wood, Noel R. Krieg and G. Briggs Phillips, eds., American Society for Microbiology, Washington, D.C., 1994, or in Brock (supra).

Yarrowia lipolytica

Yarrowia lipolytica strain ATCC® 20362™ is available from the American Type Culture Collection (Manassas, Va.). Yarrowia lipolytica strain Y2224 is a URA3⁻ version of Yarrowia lipolytica strain ATCC® 20362™. The generation of Yarrowia lipolytica strain Y2224 is described in U.S. Pat. No. 8,143,476. Briefly, Yarrowia lipolytica ATCC® 20362™ cells from a YPD agar plate (1% yeast extract, 2% bactopeptone, 2% glucose, 2% agar) were streaked onto a minimal media plate (75 mg/L each of uracil and uridine, 6.7 g/L YNB with ammonia sulfate, without amino acid, and 20 g/L glucose) containing 250 mg/L 5-FOA (5-fluorouracil-6-carboxylic acid monohydrate; Zymo Research). Plates were incubated at 28° C. and four of the resulting colonies were patched separately onto minimal media (MM) plates containing 200 mg/mL 5-FOA and MM plates lacking uracil and uridine to confirm uracil Ura3 auxotrophy.

### Example 1

### Construction of Genetic Cassette for β-Carotene Production in Yarrowia Lipolytica

Production of β-carotene requires the expression of four genes; namely crtE, crtB, crtI and crtY (Table 2) which convert farnesyl diphosphate (FPP) to β-carotene (BC) through the formation of geranylgeranylpyrophosphate (GGPP), phytoene and lycopene, respectively in Yarrowia lipolytica (FIG. 1). The genes were selected from Enterobacteriaceae bacterium DC413 (U.S. Patent Application Publication No. 2012-0142082 A1) and codon-optimized for maximal expression in Yarrowia lipolytica (see U.S. Pat. No. 7,125,672 to Picataggio et al.).

TABLE 2
Enzymes responsible for the conversion of farnesyl diphosphate
(FPP) to β-carotene.

| Conversion step | Enzyme | Gene |
|---|---|---|
| FPP to GGPP | GGPP synthase | crtE |
| GGPP to Phytoene | Phytoene synthase | crtB |
| Phytoene to Lycopene | Phytoene desaturase | crtI |
| Lycopene to β-Carotene | Lycopene cyclase | crtY |

Plasmid pZKLeuN-6EP (SEQ ID NO: 1; see U.S. Patent Application Publication No. 2012-0142082A1) based integration vector pYcrtEBI (SEQ ID NO: 2) was taken to clone GPAT promoter (SEQ ID NO: 19), the coding region of the crtY gene (SEQ ID NO: 17), and PEX16-3′ terminator (SEQ ID NO: 20). The second amino acid Thr (T) of CrtY was changed to Asp (D) in the codon-optimized sequence to accommodate NcoI site (CCATGG) for a subsequent four-piece ligation. A NotI site was introduced after the stop codon in the codon-optimized gene. The codon-optimized crtY was produced by GenScript Corp. (Piscataway, N.J.) and provided in the high-copy vector pUC57 (GENBANK® Accession No. Y14837). The GPAT promoter was PCR-amplified from pZKUGPE1S (SEQ ID NO: 3) using primers SKS001 (SEQ ID NO: 33) and SKS002 (SEQ ID NO: 34) (Table 3). Similarly, PEX16-3′ terminator (SEQ ID NO: 20) was PCR amplified from pZGDT-CPP using primers SKS007 (SEQ ID NO:35) and SKS008 (SEQ ID NO: 36) (Table 3). PCR products were gel purified using BIO101 GENECLEAN® kit (BIO 101, Vista, Calif.). Plasmid pYcrtEBI (SEQ ID NO: 2) was digested with PacI/EcoRI and the fragment was gel purified. GPAT promoter was digested with PacI/NcoI and the fragment was gel purified. The terminator was digested with NotI/EcoRI and the fragment was gel purified. A four-way ligation was used to assemble the pYcrtEBI vector backbone, GPAT promoter and PEX16-3′ promoter.

TABLE 3
List of primers used to PCR amplify GPAT and
PEX16-3′.

| | | | Sequence |
|---|---|---|---|
| Primer | Description | Template | (5′ to 3′) |
| SKS001 | F-PacI-GPAT | pZKUGPE1S-P | ACTTTAATTAACGATG |
| | | | CGTATCTGTGGGACAT |
| | | | GTGG |
| | | | (SEQ ID NO: 33) |
| SKS002 | R-NcoI-GPAT | pZKUGPE1S-P | TCACCATGGGTTAGCG |
| | | | TGTCGTGTTTTTGTTG |
| | | | TG |
| | | | (SEQ ID NO: 34) |
| SKS007 | F-NotI- | pZGDT-CPP | ACTGCGGCCGCATTGA |
| | PEX16-3′ | | TGATTGGAAACACACA |
| | | | CATG |
| | | | (SEQ ID NO: 35) |
| SKS008 | R-EcoRI- | GDT-CPP | ACTGAATTCAAGGCGT |
| | PEX16-3′ | | TGAAACAGAATGAGCC |
| | | | (SEQ ID NO: 36) |

E. coli XL2 Blue (Agilent Technologies, Santa Clara, Calif.) was transformed with the ligation mixture and plated on LB with ampicillin (Amp). Plasmids were isolated from about 20 Amp resistant colonies and digested to confirm the right clone pYcrtEBI::GPAT-crtY-PEX16-3′ (referred to as plasmid “pYcrtEBIY”; SEQ ID NO: 4; FIG. 3). The genes (promoter, coding sequence and terminator) are provided in Table 4.

TABLE 4
Genes, promoters and terminators in plasmid pYcrtEBIY.

| Gene | Promoter | Coding Sequence | Terminator |
|---|---|---|---|
| 1 | FBAIN | crtE | LIP1-3′ |
| | (SEQ ID NO: 7) | (SEQ ID NO: 5) | (SEQ ID NO: 8) |
| 2 | GPD Pro + Intron | crtB | LIP2-3′ |
| | (SEQ ID NO: 11) | (SEQ ID NO: 9) | (SEQ ID NO: 12) |
| 3 | EXP | crtI | OCT |
| | (SEQ ID NO: 15) | (SEQ ID NO: 13) | (SEQ ID NO: 16) |
| 4 | GPAT | crtY | PEX16-3′ |
| | (SEQ ID NO: 19) | (SEQ ID NO: 17) | (SEQ ID NO: 20) |

### Example 2

### Construction of Yarrowia Lipolytica Strains for the Production β-Carotene

Plasmid pYcrtEBIY (SEQ ID NO: 4) was digested with SphI/AscI and the 13.2 kb crtE-crtB-crtI-URA3-crtY fragment was gel purified. This fragment contained genes for the conversion of FPP until β-carotene. This fragment was used to transform Y. lipolytica Y2224 host and selected on minimal media plate without uracil. (Yarrowia lipolytica Y2224 is a URA3⁻ derivative of Yarrowia lipolytica ATCC® 20362™; available from the American Type Culture Collection, Manassas, Va.). About 200 yellow color colonies were screened and about 30 colonies were selected for HPLC analysis. The strains produced β-carotene with the accumulation of phytoene and lycopene as intermediates (Table 5). Y. lipolytica strain BC9A was chosen for further analysis.

TABLE 5
β-Carotene producing Y. lipolytica strain performance.

| | Phytoene | Lycopene | β-Carotene |
|---|---|---|---|
| Strain | (ppm) | (ppm) | (ppm) |
| BC 6 | 44 | 95 | 52 |
| BC 1A | 24 | 82 | 29 |
| BC 2A | 19 | 52 | 37 |
| BC 3A | 21 | 65 | 40 |
| BC 4A | 34 | 84 | 52 |
| BC 5A | 6 | 34 | 15 |
| BC 6A | 20 | 53 | 34 |
| BC 7A | 33 | 115 | 41 |
| BC 8A | 48 | 114 | 53 |
| BC 9A | 58 | 121 | 61 |
| BC 10 | 21 | 66 | 36 |
| BC 11 | 17 | 39 | 38 |
| BC 12 | 32 | 78 | 62 |
| BC 13 | 12 | 77 | 14 |
| BC 14 | 8 | 38 | 20 |
| BC 15 | 39 | 104 | 73 |
| BC 16 | 31 | 71 | 33 |
| BC 17 | 33 | 68 | 36 |
| BC 18 | 30 | 71 | 41 |
| BC 19 | 30 | 63 | 40 |
| BC 20 | 83 | 108 | 71 |
| BC 21 | 26 | 63 | 11 |
| BC 22 | 37 | 120 | 38 |
| BC 23 | 33 | 92 | 69 |
| BC 24 | 19 | 57 | 39 |
| BC 25 | 10 | 14 | 98 |
| BC 26 | 34 | 120 | 41 |
| BC 27 | 35 | 90 | 52 |
| BC 28 | 46 | 3 | 12 |
| BC 29 | 9 | 63 | 40 |
| BC 30 | 13 | 42 | 40 |

### Example 3

### HPLC Method Development for Analysis of Carotenoids

The HPLC method was developed for the separation of astaxanthin and its intermediates based upon the published report (Cunningham Jr. F and Gantt E, The Plant Journal, 2005, 41: 478-492). Standard compounds were procured from CaroteNature GmbH (Ostermundigen, Switzerland). All the peaks were confirmed by taking mass fragmentation pattern. The HPLC conditions are mentioned in Table 6 and Table 7.

TABLE 6
HPLC column and mobile phase.

| | |
|---|---|
| Column | SUNFIRE ™ C18 250 mm × 4.6 mm: 5 um (Waters |
| | Corporation, Milford Massachusetts) |
| Mobile Phase A | Acetonitrile:Water:Triethylamine (90:10:0.1 V/V) |
| Mobile Phase B | 100% Ethyl acetate |
| Column Temp | 25° C. |
| Sample Temp | 4° C. |
| Wavelength | 210 nm-700 nm |
| Flow | 1.0 mL/min |

TABLE 7
Gradiant of the mobile phase in HPLC.

| Time (min) | % A | % B |
|---|---|---|
| 0.01 | 90 | 10 |
| 15 | 75 | 25 |
| 18.0 | 50 | 50 |
| 23.0 | 20 | 80 |
| 30.0 | 75 | 25 |
| 40.0 | 90 | 10 |

Astaxanthin and nine intermediates of the pathway were well separated in a single HPLC run (Table 8, FIG. 2).

TABLE 8
Retention time of astaxanthin and related analytes in HPLC.

| Sample No. | Analyte | Retention time (min) |
|---|---|---|
| 1 | Astaxanthin | 7.54 |
| 2 | Adonixanthin | 7.81 |
| 3 | Zeaxanthin | 8.10 |
| 4 | Adonirubin | 8.49 |
| 5 | Canthaxanthin | 14.38 |
| 6 | β-Cryptoxanthin | 22.71 |
| 7 | Echinenone | 23.33 |
| 8 | Lycopene | 24.78 |
| 9 | β-Carotene | 26.11 |
| 10 | Phytoene | 26.76 |

Standard curves were generated using authentic compounds for the quantitation of various carotenoids. Astaxanthin solution in DMSO was used to generate standard curve by diluting it in acetone:petroleum ether 1:1 with 2% DMSO solution.
Yarrowia lipolytica cells were grown in Fermentation Medium (FM) with the following composition:

| | | |
|---|---|---|
| Yeast nitrogen base (w/o | 6.7 | g/L |
| AAs, w/AS) | | |
| Yeast Extract | 5 | g/L |
| KH₂PO₄ | 6 | g/L |
| K₂HPO₄ | 2 | g/L |
| MgSO₄•7H₂O | 1.5 | g/L |
| Thiamine hydrochloride | 1.5 | mg/L |
| Water | to 960 | mL |

The medium was sterilized by autoclaving followed by addition of 40 mL 50% sterile glucose solution resulting 2% final glucose concentration. Yarrowia lipolytica strain was grown in 25 mL FM in a 250-mL flask at 30° C. in a rotary shaker at 250 rpm. After 2 days of growth, 2 mL of cell culture was harvested by centrifugation and the cell pellet was extracted using the method described below for carotenoid analysis. At the same time, 5 mL culture was used for dry cell weight measurement. Extraction protocol was developed based upon the method mentioned in Pat Pub No.: U.S. Patent Application Publication No. 2012-0142082 A1 with some modifications as mentioned below.

The cells pellet was chilled in ice and 0.5 mm glass beads were added to the tube. 1 mL pre-chilled acetone:petroleum ether solvent (1:1 mixture) with 0.01% butylated hydroxytoluene and 2% dimethyl sulfoxide was added to tube. The mixture was agitated in a BEADBEATER™ for 2 minutes. The mixture was centrifuged for 1 min at 13,000 rpm and the supernatant was transferred into a new tube. The process was repeated once and the supernatant was added to the first supernatant. The collected supernatant was filtered using 0.2 μm DMSO-safe acrodisc syringe filter (Pall Corporation, Cat No. #4433). The carotenoids extract was analyzed by HPLC as mentioned above.

### Example 4

### Selection of β-Carotene Ketolase (crtW) and β-Carotene Hydroxylase (crtZ) Genes

The conversion of β-carotene to astaxanthin involves two enzymes, i.e., β-carotene ketolase and β-carotene hydroxylase. These two enzymes put two keto- and two hydroxyl-group in β-carotene. This conversion is typically inefficient due to the possibility of eight different intermediates (FIG. 1). Therefore, a need existed to identify a combination of CrtW β-carotene ketolase) and CrtZ (β-carotene hydroxylase) enzymes which can convert β-carotene to astaxanthin efficiently without the accumulation of the above said intermediates.

The coding sequence of the β-carotene ketolase gene crtW (GENBANK® Accession No. AY860820.1; SEQ ID NO: 21) from Chlamydomonas reinhardtii (Zhong et al. 2011 J. Exp. Botany., 62: 3659-3669) was selected to be used in combination with the coding sequence of a β-carotene hydroxylase gene crtZ (GENBANK® Accession No. ABC50108.1; SEQ ID NO: 25) from Brevundimonas vesicularis (Tao et al., Gene, 2006 379:101-108). To accommodate NcoI site for the four-piece ligation, amino acid Ala, i.e. GCC codon was introduced after ATG start codon of the codon-optimized crtZ from B. vesicularis. Similarly, the coding sequence of a β-carotene hydroxylase gene crtZ (GENBANK® Accession No. NP_{—}194300; SEQ ID NO: 29) from Arabidopsis thaliana (Sun et al., 1996, J. Biol. Chem., 271:24349-24352) was selected to be used in combination with the Chlamydomonas reinhardtii β-carotene ketolase. The codon-optimized crtZ from A. thaliana was modified as follows: two amino acids Met-Ala were taken from the predicted sequence tag (GENBANK® Accession No. F13822) and added to N-terminus of the 294 amino acid sequence of crtZ (GENBANK® Accession No. U58919), which resulted in NcoI site for subsequent four-piece ligation.

### Example 5

### Construction of CrtW-CrtZ Integration Cassettes

The coding sequence of crtW (SEQ ID NO: 21) from C. reinhardtii (designated as crtW_{Cr}) was codon optimized for maximal expression in Y. lipolytica. The source of the coding sequence, promoter and terminator for cloning crtW_{Cr} in pZKLeuN-6EP (SEQ ID NO: 1) is shown in Table 9.

TABLE 9
Source of the DNA fragments for the cloning of crtW_{Cr}

| | | Desired | |
|---|---|---|---|
| | Restriction | fragment | |
| Source plasmid | enzymes | (bp) | Identity |
| pZKIeuN-6EP | BgIII-Swal | 8639 bp | pZKIeuN-6EP |
| | | | backbone |
| pZKIeuN-6EP | BgIII-Ncol | 989 bp | FBAIN promoter |
| pZKIeuN-6EP | Notl-Swal | 332 bp | LIP1-3′ |
| pUC57-crtW_{Cr} | Ncol-Notl | 789 bp | crtW_{Cr} fragment |

The coding sequence for crtW_{Cr} (SEQ ID NO: 21) was cloned in pZKIeuN-6EP integration vector under the control of FBAIN promoter (SEQ ID NO: 23) and LIP1-3′ (SEQ ID NO: 24) was used as terminator, resulting in pYcrtW_{Cr}. The plasmid was confirmed by restriction digestion with BglII/SwaI and gel analysis, resulting in two bands of 2105 and 8639 bps. A four piece ligation was used to construct pYcrtW_{Cr}-crtZ_{Bv} (FIG. 5; SEQ ID NO: 37; Table 10) and pYcrtW_{Cr}-crtZ_{At} (FIG. 4; SEQ ID NO: 38; Table 11).

TABLE 10
Source of the DNA fragments for the construction of pYcrtW_{Cr}-
crtZ_{Bv}.

| Source plasmid | Restriction enzymes | Identity |
|---|---|---|
| pYcrtW_{Cr} | Clal/Pmel | pYcrtW_{Cr} backbone |
| pZKIeuN-6EP | Clal/Ncol | GPD promoter |
| pZKIeuN-6EP | Notl-Pmel | PEX16-3′ terminator |
| pUC57-crtZ_{Bv} | Ncol/Notl | crtZ_{Bv} fragment |

TABLE 11
Source of the DNA fragments for the construction of pYcrtW_{Cr}-
crtZ_{At}.

| Source plasmid | Restriction enzymes | Identity |
|---|---|---|
| pYcrtW_{Cr} | Clal/Pmel | pYcrtW_{Cr} backbone |
| pZKIeuN-6EP | Clal/Ncol | GPD promoter |
| pZKIeuN-6EP | Notl-Pmel | PEX16-3′ terminator |
| pUC57-crtZ_{At} | Ncol/Notl | crtZ_{At} fragment |

The synthetic genes were produced by GenScript Corp. (Piscataway, N.J.) and provided in the high-copy vector pUC57 (Gen Bank® Accession No. Y14837).

### Example 6

### Construction of Y. Lipolytica Strains Producing Astaxanthin

β-Carotene producing Y. lipolytica strain BC9A (Example 3) was chosen to introduce crtW-crtZ combinations. First, the URA3 marker of BC9A strain was removed according to the method described in US Patent Application Publication No. US 2012-0142082A1. Next, the crtW-crtZ-URA3 cassette was introduced in BC9A URA3⁻ host and plated on minimal media plate without uracil supplementation. About 600 colonies for each set were screened on plate for yellow-red color colonies for possible astaxanthin production. Y. lipolytica BC9A URA3⁻ strains which received crtZ_{Ar}-crtW_{Cr} were designated as the AX150 series and strains which received crtW_{Cr}-crtZ_{Bv} were designated as AX250 series. About 30 yellow-red colonies of each sets were chosen for carotenoid quantitation.

### Example 7

### Production of Astaxanthin in Y. Lipolytica without Measurable Concomitant Accumulation of Ketolated- or Hydroxylated-β-Carotene Intermediates

Y. lipolytica strains were grown in fermentation media (FM composition mentioned in EXAMPLE 2) and samples were taken at 48 hr to determine the carotenoid content of each strain. As shown in Table 12, the strains produce axtaxanthin without any detectable amount of ketolated- or hydroxylated-β-carotene compounds such as adonixanthin, zeaxanthin, adonirubin, cantaxanthin, β-cryptoxanthin and echienone.

The detection limit of ketolated- and hydroxylated-β-carotene intermediates were calculated using β-carotene as standard and the detection limit in the HPLC was 0.00825 ppm. Now, under the extraction process where about 10 mg dcw of Yarrowia cells were used to extract carotenoids with 2 mL of solvent, the detection limit would be <2 ppm of dcw.

TABLE 12
Astaxanthin producing Y. lipolytica strain performance

| | | | Lyco- | β- | |
|---|---|---|---|---|---|
| | Gene | Phytoene | pene | Carotene | Astaxanthin |
| Strain | combination | (ppm) | (ppm) | (ppm) | (ppm) |
| AX-155 | crtZ_{At}-crtW_{Cr} | 59 | 356 | 147 | 30 |
| AX-157 | crtZ_{At}-crtW_{Cr} | 55 | 299 | 92 | 263 |
| AX-159 | crtZ_{At}-crtW_{Cr} | 51 | 285 | 78 | 221 |
| AX-160 | crtZ_{At}-crtW_{Cr} | 97 | 334 | 209 | 0 |
| AX-165 | crtZ_{At}-crtW_{Cr} | 48 | 271 | 89 | 276 |
| AX-167 | crtZ_{At}-crtW_{Cr} | 95 | 348 | 95 | 68 |
| AX-173 | crtZ_{At}-crtW_{Cr} | 58 | 304 | 84 | 249 |
| AX-176 | crtZ_{At}-crtW_{Cr} | 67 | 330 | 103 | 81 |
| AX-180 | crtZ_{At}-crtW_{Cr} | 123 | 275 | 84 | 193 |
| AX-252 | crtW_{Cr}-crtZ_{Bv} | 114 | 398 | 69 | 185 |
| AX-257 | crtW_{Cr}-crtZ_{Bv} | 91 | 363 | 217 | 0 |
| AX-258 | crtW_{Cr}-crtZ_{Bv} | 112 | 384 | 72 | 183 |
| AX-262 | crtW_{Cr}-crtZ_{Bv} | 68 | 327 | 73 | 148 |
| AX-265 | crtW_{Cr}-crtZ_{Bv} | 128 | 450 | 80 | 297 |
| AX-267 | crtW_{Cr}-crtZ_{Bv} | 114 | 385 | 72 | 181 |
| AX-271 | crtW_{Cr}-crtZ_{Bv} | 104 | 363 | 62 | 173 |
| AX-275 | crtW_{Cr}-crtZ_{Bv} | 113 | 385 | 77 | 169 |
| AX-279 | crtW_{Cr}-crtZ_{Bv} | 91 | 353 | 66 | 175 |
| AX-282 | crtW_{Cr}-crtZ_{Bv} | 110 | 381 | 67 | 202 |

Astaxanin-producing Y. lipolytica strains AX165 and AX265 produced 276 and 297 ppm dry cell weight (dcw) astaxanthin without accumulation of any detectable amount (i.e., less than 2 ppm) of ketolated- or hydroxylated-β-carotene compounds (Tables 12 and 13).

TABLE 13
Astaxanthin-Producing Strains AX165 and AX265.

| | Strain AX165 | Strain AX265 |
|---|---|---|
| Carotenoid | (ppm) | (ppm) |
| Astaxanthin | 276 | 297 |
| Adonixanthin | ND | ND |
| Zeaxanthin | ND | ND |
| Adonirubin | ND | ND |
| Canthaxanthin | ND | ND |
| β-Cryptoxanthin | ND | ND |
| Echinenone | ND | ND |
| Lycopene | 271 | 450 |
| β-Carotene | 89 | 80 |
| Phytoene | 48 | 128 |

ND = not detected.
Limit of detection = <2 ppm

What is claimed is:

1. A recombinant microbial host cell comprising:
a. a set of β-carotene biosynthesis pathway genes;
b. at least one expressible genetic construct encoding the β-carotene ketolase from Chlamydomonas reinhardtii; and
c. at least one expressible genetic construct encoding a carotenoid hydroxylase selected from Brevundimonas sp., Arabidopsis thaliana or a combination thereof;
wherein the recombinant microbial host cell produces astaxanthin from β-carotene and does not concomitantly accumulate a significant amount of any one of the following ketolated and/or hydroxylated carotenoid intermediates: adonixanthin, zeaxanthin, adonirubin, echinenone, 3-hydroxyechinenone, 3′-hydroxyechinenone, canthaxanthin or β-cryptoxanthin; wherein the ratio of astaxanthin to any one of the ketolasted and/or hydroxylated carotenoid intermediates as measured by dry cell weight is at least 75:1, preferably at least 100:1, more preferably at least 125:1, and most preferably at least 150:1.

2. The recombinant microbial host cell of claim 1, wherein one or more of the set of β-carotene biosynthesis pathway genes present are foreign genes.

3. The recombinant microbial host cell of claim 1, where the set of β-carotene biosynthesis pathway genes are endogenous to the recombinant microbial host cell.

4. The recombinant microbial host cell of claim 1, 2 or 3 wherein the recombinant microbial host cell is a prokaryotic cell or eukaryotic cell.

5. The recombinant microbial host cell of claim 4 where the prokaryotic cell is a recombinant bacterial cell.

6. The recombinant microbial host cell of claim 4 where the eukaryotic cell is a recombinant fungal cell.

7. The recombinant microbial host cell of claim 6 where the recombinant fungal cell is a yeast.

8. The recombinant microbial host cell of claim 7 wherein the yeast is selected form the genera Phaffia, Xanthophyllomyces, Saccharomyces, Thraustochytrium, Yarrowia, and Labyrinthula.

9. The recombinant microbial host cell of claim 8 wherein the yeast is Yarrowia lipolytica.

10. The recombinant microbial host cell of claim 1, where the β-carotene ketolase from Chlamydomonas reinhardtii comprises an amino acid sequence having at least 95% identity to SEQ ID NO: 22.

11. The recombinant microbial host cell of claim 1, where the β-carotene ketolase from Chlamydomonas reinhardtii comprises an amino acid sequence SEQ ID NO: 22.

12. The recombinant microbial host cell of claim 10 or claim 11 wherein the carotenoid hydroxylase comprises an amino acid sequence having at least 95% identity to SEQ ID NO: 26 or SEQ ID NO 30.

13. The recombinant microbial host cell of claim 12 wherein the carotenoid hydroxylase comprises an amino acid sequence of SEQ ID NO: 26 or SEQ ID NO: 30.

14. The recombinant microbial host cell of claim 1 wherein
a. the β-carotene ketolase comprises amino acid sequence SEQ ID NO: 22; and
b. the carotenoid hydroxylase comprises amino acid sequence SEQ ID NO: 26 or SEQ ID NO: 30.

15. A method to produce astaxanthin comprising:
a. providing the recombinant microbial host cell of any one of claims 1-14; and
b. growing the recombinant microbial host cell whereby astaxanthin is produced.

16. A method to produce an animal feed comprising astaxanthin comprising:
a. providing the astaxanthin produced in claim 15;
b. adding an effective amount of the astaxanthin to an animal feed whereby an animal feed comprising astaxanthin is produced.

17. A method to pigment the muscle tissue of an animal comprising:
a. providing the animal feed comprising astaxanthin of claim 16;
b. feeding an animal the animal feed comprising astaxanthin whereby the muscle tissue of the animal is pigmented by the astaxanthin present in the animal feed.

18. The method of claim 17, wherein the animal is a fish or shellfish.

19. The method of claim 18 wherein the fish is a member of the family Salmonidae.

20. The method of claim 19 wherein the fish is salmon.
